SEQUENCE LISTING (4 sequences)

SEQ ID NO: 1 - protein, 21 aa, Homo sapiens
SSMKLSFRARGYSFRGPGPQL

SEQ ID NO: 2 - protein, 21 aa, Homo sapiens
SSMKLSFRARAYGFRGPGPQL

SEQ ID NO: 3 - protein, 21 aa, Homo sapiens
SSMKLSFRARAYGFRGPGLQL

SEQ ID NO: 4 - protein, 21 aa, Homo sapiens
SSMKLSFRARAYSFRGPGPQL

(19) United States
(12) Patent Application Publication
(10) Pub. No.: US 2011/0166066 A1
(43) Pub. Date: Jul. 7, 2011

(54) CATESTATIN (CST) AND ITS VARIANTS FOR TREATMENT OF CARDIOVASCULAR AND METABOLIC DISORDERS

(76) Inventors: Sushil Mahata, San Diego, CA (US); Bruno Tota, Naples (IT); Daniel O'connor, San Diego, CA (US); Gautam Bandyopadhyan, San Diego, CA (US); Rudolf Kirchmair, Telfs (AT)

(21) Appl. No.: 12/736,842

(22) PCT Filed: May 8, 2009
(86) PCT No.: PCT/US2009/002865

§ 371 Date: Feb. 1, 2011

Related U.S. Application Data
(60) Provisional application No. 61/126,913, filed on May 8, 2008.

(51) Int. Cl. A61K 38/17; C07K 14/47; C07H 21/00; C07K 16/18; C12Q 1/02

(52) U.S. Cl. 514/6.7; 530/326; 514/21.4; 536/23.5; 530/388.2; 530/389.8; 514/6.9; 514/6.8; 514/15.7; 514/16.4; 435/29

(57) ABSTRACT

Circulating levels of catestatin (Cts: human chromogranin A352-372) decrease in the plasma of patients with essential hypertension. Genetic ablation of the chromogranin A (Chga) gene in mice increases blood pressure and pre-treatment of Chga-null mice with Cts prevents blood pressure elevation, indicating a direct role of Cts in preventing hypertension. This notable vasoreactivity prompted us to test the direct cardiovascular effects and mechanisms of action of wild-type Cts (WT-Cts) and naturally occurring human variants (G364S-Cts and P370L-Cts) on myocardial and coronary functions. The cardio-inhibitory influence exerted on basal mechanical performance and the counter-regulatory action against beta-adrenergic and ET-1 stimulations, point to Cts as a novel cardiac modulator, able to protect the heart against excessive sympathochromaffin over-activation, e.g. hypertensive cardiomyopathy.

### CROSS REFERENCE TO RELATED APPLICATIONS

The present invention claims benefit of priority to U.S. provisional patent application Ser. No. 61/126,913, filed on May 8, 2008, the entire contents of which are incorporated herein by reference in its entirety.

### STATEMENT AS TO FEDERALLY SPONSORED RESEARCH

This invention was made with Government support under Grant No. DA011311, awarded by the National Institutes of Health. The government has certain rights in the invention.

### FIELD OF INVENTION

The present invention relates generally to the field of biochemistry and medicine relates to the polypeptide catestatin and its variants, and particularly to any and all portions the wild-type catestatin (“WT-CST”) and naturally occurring variants that act as a cardio-depressant agent and/or are able to treat hypertensive individuals. Variation of Gly364 to Ser within the catestatin domain is designated as Gly364Ser (“Gly364Ser”), and variation of Pro370 to Leu within the catestatin domain is designated as Pro370Leu (“Pro370Leu”).

### INTRODUCTION

Throughout this application various publications are referenced, many referenced by numbers in parenthesis. Full citations for these publications are provided later in this application. All of the disclosures of these publications are hereby incorporated by reference, in their entirety, in this application. Citation of these documents is not intended as an admission that any of the material is pertinent prior art. All statements as to the date or representation as to the contents of these documents is based on the information available to the Applicants and does not constitute any admission as to the correctness of the dates or contents of these documents.

Chromogranin A (CgA), an index member of the chromogranin/secretogranin protein family, is a secretory pro-protein (1-3) that gives rise to several peptides of biological importance including the dysglycemic hormone pancreastatin (4), vasodilator vasostatin (5), and the catecholamine release inhibitory peptide catestatin (CST: human CgA₃₅₂₋₃₇₂, bovine CgA₃₄₄₋₃₆₄) (6) (7) (8) (9) (10, 11). CgA is co-stored with catecholamines and its co-release documents exocytosis as the mechanism of physiologic catecholamine release in humans. The basal plasma CgA level not only correlates with sympathetic tone, but also shows highest heritability. Plasma CgA levels increase in patients with essential hypertension. The serum levels of CgA are also elevated in patients with heart failure. CST is decreased not only in patients with essential hypertension but also in normotensive subjects with a family history of hypertension (12). People with such a family history demonstrate increased epinephrine secretion in addition to diminished CST (12), implicating an inhibitory effect of catestatin on chromaffin cells in vivo. Consistent with human studies, genetic ablation of Chga gene results in high blood pressure in mice (13). High blood pressure in Chga-/- mice can be rescued either by pre-treatment with CST or introduction of the human gene in the Chga-/- background utilizing bacterial artificial chromosome (BAC) transgenic technology. Furthermore, low CST levels predict augmented adrenergic responses to stressors, suggesting that a reduction in CST may increase the risk of hypertension.

To understand human genetic variation at CHGA and its CST fragment, O'Connor's group systematically searched for polymorphism at the CHGA locus, and reported 3 naturally occurring amino acid substitution variants in the catestatin region (9). Although two of the catestatin variants (Pro370Leu & Arg374Gln) were reported to be relatively rare (minor allele frequencies 0.3-0.6%), one variant, Gly364Ser (S352SMKLSFRARGYS364FRGPGPQL372: SEQ ID NO. 1; position in the mature CgA protein) showed an allele frequency of ˜3-4%. These CST variants displayed differential potencies toward inhibition of nicotinic cholinergic agonist-evoked catecholamine secretion from sympathochromaffin cells in vitro, in the following rank order of potency Pro370Leu>wild-type (WT)>Gly364Ser>Arg374Gln (11). In vivo, human carriers of the 364Ser allele had profound alterations in autonomic activity, in both the parasympathetic and sympathetic branches, and may be protected against the future development of hypertension, especially in males (14).

The working heart and the arterial system are such close functional complements that the analysis of their dynamic interaction represents an obligatory step in the integral understanding of cardiovascular homeostasis both under normal and abnormal (hypertension) conditions. Nothing is known regarding the influence of catestatin on the pumping heart. Therefore, we looked at the effects of catestatin and its naturally occurring variants on the inotropic and lusitropic functions using the Langendorff perfused rat heart. The results provide for the first time an insight into the role of catestatin as a cardiac modulator, adding new aspects to the anti-hypertensive activity of this peptide.

### BACKGROUND

CST is the most potent endogenous nicotinic cholinergic antagonist. The structure-function relationship of this peptide has been established and human studies indicate that CST is low, not only in hypertensive individuals, but also in individuals with family history positive for hypertension. Pre-treatment with CST lowers the high blood pressure in CgA null mice to normal levels. It appears from the recent human data that the Gly364Ser variant of catestatin prevents humans from developing hypertension. The present invention demonstrates that catestatin acts on the heart and dramatically reduces the cardiac contractility. These effects are mediated via the nitric oxide/cyclic GMP pathway.

### SUMMARY OF THE INVENTION

The present invention relates generally to the polypeptide catestatin and its variants, and particularly to any and all portions the wild-type catestatin (“WT-Cts”) and naturally occurring variants (“Pro370Leu” and “Gly364Ser”) that act as a cardio-depressant agent and/or are able to treat hypertensive individuals. The amino acid sequences of these polypeptides are as follow:

| SEQ ID NO. 2 | | |
|---|---|---|
| 1. | CgA₃₅₂₋₃₇₂ (WT-CST): | SSMKLSFRARAYGFRGPGPQL: |
| SEQ ID NO. 3 | | |
| 2. | Pro370Leu-CST: | SSMKLSFRARAYGFRGPGLQL: |
| SEQ ID NO. 4 | | |
| 3. | Gly364Ser-CST: | SSMKLSFRARAYSFRGPGPQL: |

The present invention also includes the nucleic acid sequences of the DNA and RNA molecules, which ultimately encode for these polypeptides. The invention teaches a method to decrease cardiac contractility (by >60%) using an endogenous peptide catestatin and its naturally occurring variants. CST and its naturally occurring human variants (Gly364Ser or Pro370Leu) act as myocardial modulators in the mammalian to almost abolish isoproterenol or endothelin-induced changes in cardiac parameters. The invention can be used as a cardio-depressant agent and to treat hypertensive individuals.

CST is decreased not only in patients with essential hypertension but also in normotensive subjects with a family history of hypertension. Genetic ablation of Chga gene resulted in high blood pressure in mice and pre-treatment with catestatin rescued mice from such high blood pressure indicating a role of CST in the development of hypertension.

### BRIEF DESCRIPTION OF THE DRAWINGS

FIG. 1. Dose-dependent response curves of WT-CST (11 to 200 nM) on (A) heart rate (HR), (B) Myocardial parameters: LVP, RPP, and +(LVdP/dT)max), (C) lusitropic parameters: −(LVdP/dT)max], and T/−t and (D) CP on Langendorff perfused rat heart preparation. For abbreviations and basal values see results. Percentage changes were evaluated as means±SEM of 8 experiments. Significance of difference from control values was done by one-way ANOVA followed by Bonferroni's post-hoc test: α: p<0.05; β: p<0.01; γ: p<0.001.

FIG. 2. Effects of WT-CST (110 nM) before and after treatment with either Nadolol (10 nM), or Phentolamine (100 nM) or Atropine (10 nM) on LVP, +(LVdP/dT)max and −(LVdP/dT)max on the isolated and Langendorff perfused rat heart. Significance between the control and WT-CST-treated values were done by one-way ANOVA (p<0.0001) followed by Bonferroni's post-hoc-test (n=5): p value α; Kreb's buffer vs WT-Cts; p value β: WT-CST vs inhibitors.

FIG. 3. Effects of WT-CST (110 nM) alone and WT-CST in presence of PTx on LVP, +(LVdP/dT)max and −(LVdP/dT)max. Percentage changes were evaluated as means±SEM of 5 experiments. Significance between the control and WT-CST-treated values were done by one-way ANOVA (p<0.001) followed by Bonferroni's post-hoc-test: p value α: Buffer vs WT-CST; p value β: Wt-CST vs PTx.

FIG. 4. Effects of WT-CST (110 nM) alone and WT-CST in presence of L-NMMA, or PTIO, or ODQ or KT5823 on LVP, +(LVdP/dT)max and −(LVdP/dT)max. Significance between the control and WT-CST-treated values were done by one-way ANOVA (p<0.05) followed by Bonferroni's post-hoc-test (n=6): Bonferroni test: p value β: CST vs CST plus L-NMMA.

FIG. 5. Immunoblot analysis of total and phosphorylated PLN (A), AKT (B), ERK1/2 (C) and GSK-3β (actin instead of total) (D) in control and WT-CST-treated hearts. 10 μg of SR membranes were used for detection of total (T-PLN) and phosphorylated (P-PLN) PLN. P-PLN was normalized to T-PLN. 200 μg of cytosolic protein was used for immunoblot analysis for phosphorylated-Akt-Ser473 (P-Akt), total-Akt (T-Akt), phosphorylated GSK-3β, and actin. 100 μg of cytosolic protein was used for immunoblot analysis for phosphorylated-ERK1/2 (P-ERK1/2) and total-ERK (T-ERK1/2). The phosphorylated proteins were normalized either to their corresponding non-phosphorylated forms or to actin. Control (n=3); WT-CST (110 nM; n=4).

FIG. 6. Concentration-dependent response curves of G364S-CST (11 to 200 nM) on HR, on myocardial parameters (LVP, RPP and +(LVdP/dT)max) on −(LVdP/dT)max). Percentage changes were evaluated as means±SEM of 8 experiments. Significance of difference from control values was done by one-way ANOVA (p<0.05) followed by Bonferroni's post-hoc test.

FIG. 7. Concentration-dependent response curves of P370L-CST (11 to 200 nM) on HR, on myocardial parameters (LVP, RPP and +(LVdP/dT)max) on −(LVdP/dT)max). Percentage changes were evaluated as means±SEM of 8 experiments. Significance of difference from control values was done by one-way ANOVA (p<0.05) followed by Bonferroni's post-hoc test.

FIG. 8. Effects of ISO before and after treatment with either WT-Cts or P370L-Cts or G364S-Cts on LVP, +(LVdP/dT)max, CP, −(LVdP/dT)max, HTR, T/−t or +(LVdP/dT)max/−(LVdP/dT)max. For abbreviations and basal values see Results section. Percentage changes were evaluated as means±SEM of 6 experiments for each group. Significance of difference from control and ISO-treated values were done one-way ANOVA (p<0.05) followed by Bonferroni's post-hoc test. Bonferroni test: p value α: Buffer vs Iso; p value β: Iso vs Iso plus CST.

FIG. 9. The concentration-response curves of ISO-mediated stimulation on LVP of ISO (10¹⁰ to 10⁻⁶ M) alone and ISO (10⁻¹⁰ to 10⁻⁶ M) plus a single concentration of either WT-CST (11, 33 and 110 nM) or P370L-CST (165 nM) or G364S-CST (200 nM). Contraction is expressed as a percentage of LVP [baseline=0%, peak constriction by ISO and ISO plus WT-Cts=100%]. The EC₅₀ values (in log M) were: ISO alone −8.67±0.3 (r²=0.84), ISO plus WT-Cts (11 nM, or 33 nM, or 110 nM) −8.7±0.35 (r²=0.85), −7.35±0.46 (r²=0.73), −7.2±1.14 (r²=0.29), respectively, or plus P370L-Cts (165 nM) −8.51±0.46 (r²=0.76), or plus G364S-Cts (200 nM) −8.61±0.569 (r²=0.60). Comparison between groups and interaction between ISO vs catestatin peptides were done by two-way ANOVA (n=6): ISO dose: p<0.0001 (WT-CST at 11, 33, and 110 nM, G364S-CST and P370L-CST); ISO vs CST: p<0.0001 (WT-CST at 33 and 110 nM; G364S-CST and P370L-CST); Interaction: p<0.0001 (WT-CST at 33 and 110 nM), p<0.0019 (G364S-CST) and p<0.00016 (P370L-CST).

FIG. 10. ET-1 effects before and after treatment with either WT-CST or P376L-CST or G364S-CST on LVP, +(LVdP/dT)max and CP. Percentage changes were evaluated as means±SEM of 6 experiments for each group. Significance of difference from control and ET-1-treated values were done by one-way ANOVA followed by Bonferroni's post-hoc test (n=6): p<0.05. Bonferroni test: p value α: Buffer vs ET-1; p value β: ET-1 vs Cts.

FIG. 11. Schematic diagram showing the putative ET-1, ISO or CST signaling in endothelial and myocardial cells. AC: adenylate cyclase, β-AR: β-adrenergic receptor, β-ARK: β-adrenergic receptor kinase, DHPR: dihydropyridine receptor, ET_{AR}: ET receptor subtype A, eNOS: endothelial NO synthase, IP3: inositol triphosphate, NCX: Na⁺/Ca²⁺ exchangers, NO: nitric oxide, PKA: protein kinase A, AKT: protein kinase B, PLN: phospholamban, PI-3-K: phosphoinositide 3 kinase, PLC: phospholipase C, PMCA: plasma membrane Ca2+-ATPases, RyR: ryanodine receptor, SERCA: sarco(endo)plasmic reticulum Ca²⁺-ATPases, SR: sarcoplasmic reticulum. (+): stimulation, (−): inhibition, (±): no effect.

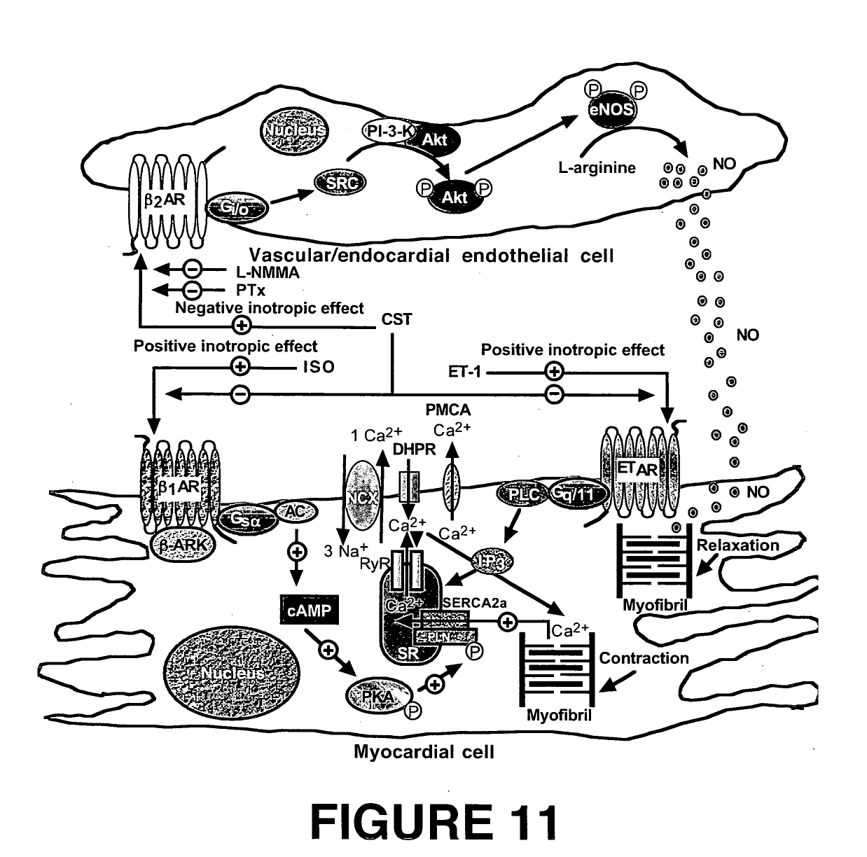

FIG. 12. Immobilization stress-induced alterations of SBP, DBP and HR and the effects of catestatin. (A) SBP during stress and recovery period. (B) SBP after supplementation with catestatin (CST: hCgA₃₅₂₋₃₇₂; 40 μg/g bw ip) or saline 30 min before stress followed by stress and recovery. (C) DBP during stress and recovery. (D) DBP after supplementation with CST (40 μg/g bw ip) or saline 30 min before stress followed by stress and recovery. (E) HR during stress and recovery period. (F) HR after supplementation with CST (40 μg/g bw IP) or saline 30 min before stress followed by stress and recovery. *: p<0.05; **: p<0.01 (comparison with “0” time point).

FIG. 13. Plasma catecholamine in WT and KO mice. (A) Plasma catecholamines under basal and after supplementation with catestatin (40 μg/g bw ip) for 30 min. p-values represent comparison between WT and KO mice. (B). Plasma catecholamine under basal and after 15 min of immobilization stress. α: comparison between control and stress; β: comparison between WT and KO mice.

FIG. 14. Baroreceptor slope after treatment with PE (0.005 μg/g bw iv) or SNP (0.05 μg/g bw iv) in unconscious WT and KO mice or after supplementation of CST (40 μg/g bw iv) in KO mice

FIG. 15. Heart rate. Graph depicting marked increase in heart rate at baseline in CgA KO mice. This increase in heart rate can be “rescued” by parenteral treatment with CST.

FIG. 16. Cardiac rate tachogram (A&B) and power spectra (C) from WT (A) and KO (B&C) mice. A costal diaphragmatic electromyogram (bandpass: 10 Hz-3 kHz) taken from a KO mouse, showing an approximate respiratory frequency of 5 Hz, corresponding to the HRV's HF band (D).

FIG. 17. Time-domain parameters of HRV such as SDNN (A) and RMSSD (B) in the WT and in the KO mice, showing catestatin reversal and improvements of constraint parameters in the KO mice. α: WT vs KO; β: Control vs CST

FIG. 18. Typical representations of return maps acquired from WT (A) and KO (B) mice. Plotted on abscissa is (RRJ) against next (RRJ+1) interval time. KO mice distinctly revealed its compact nature of point dispersion (i.e. narrow HRV variability) compared with the increased dimension of point area in the WT mice.

FIG. 19. CST induces angiogenesis in-vivo in the mouse cornea assay. A CST-containing pellet was implanted into the cornea of mice. 7 days after implantation mice were injected intravenously with FITC-labeled BS1-lectin. After 15 minutes mice were sacrificed, corneas dissected and subjected to fluorescent microscopy. CST induced growth of arteries (red arrows) originating from the limbus artery (red arrowhead), forming a capillary plexus (yellow arrow) at the bottom of the pellet and draining the blood via veins (blue arrows to the limbus vein (blue arrowhead).

FIG. 20. CST induces therapeutic angiogenesis and arteriogenesis in the mouse hind-limb ischemia model. (a) CST improves blood perfusion. Mice were subjected to hind-limb ischemia operation and were treated by injections of CST peptide or saline. Limb perfusion was measured by LDPI before and after operation and weekly thereafter for 4 weeks. Results are expressed as LDPI ratio of the operated versus the not operated leg. CST improved perfusion compared to saline 3 and 4 weeks after operation. (b) CST increases capillary density. Mice were sacrificed 4 weeks after hind-limb ischemia operation and sections from ischemic muscles were stained for capillary density by alkaline phosphatase. Values are expressed as capillaries/mm². CST significantly increased capillary density.(c) CST increases density of arteries/arterioles. 4 weeks after hind-limb ischemia operation sections from ischemic muscles were subjected to immunohistochemistry for alpha-smooth muscle actin. Values are expressed as smooth muscle actin-positive arteries or arterioles/mm². *P<0.05; **P<0.01

FIG. 21: CST effect on glucose metabolism. CST reduced pAMPK (A) and eNOS signals, raised blood glucose level in WT and CgA-KO mice (c) but reduced glucose level in high fat (60%) fed (HFD) insulin resistant mice.

Unless defined otherwise, all technical and scientific terms used herein have the same meaning as commonly understood by one of ordinary skill in the art to which this invention belongs. Where a term is provided in the singular, the inventor also contemplates the plural of that term. The nomenclature used herein and the procedures described below are those well known and commonly employed in the art. All of the disclosures of these publications are hereby incorporated by reference, in their entirety, in this application.

All headings are for the convenience of the reader and should not be used to limit the meaning of the text that follows the heading, unless so specified. Various changes and departures may be made to the present invention without departing from the spirit and scope thereof. Accordingly, it is not intended that the invention be limited to that specifically described in the specification or as illustrated in the drawings, but only as set forth in the claims.

### EXAMPLE 1

### Catestatin Acts as a Potent Cardiosuppressive Peptide

### INTRODUCTION

Chromogranin A (CgA), an index member of the chromogranin/secretogranin protein family, is a secretory pro-protein (1, 2, 15) that is endo-proteolytically processed to give rise to several peptides of biological importance including the dysglycemic hormone pancreastatin (4), vasodilator vasostatin (5), and the catecholamine release inhibitory peptide CST (6, 8, 10, 11, 16). CgA is co-stored with catecholamines and its co-release documents exocytosis as the mechanism of physiologic catecholamine release in humans (17). CST and pancreastatin have been postulated as important counter-regulatory hormones in “zero steady-state error” homeostasis (i.e. the perfect equilibrium generated by the balance between two counter regulatory hormones (18)), a role now extended also to vasostatin 1 (VS-1) in cardiac biology ((15), and references therein). The importance of CgA in cardiovascular homeostasis in man is documented by its increased plasma levels in various diseases, such as neuroendocrine tumors (18) and chronic heart failure (19) and its colocalization with BNP and over-expression in human dilated and hypertrophic cardiomyopathy (20). Basal plasma levels of CgA correlate with sympathetic tone (21) showing a high heritability (22). Plasma level of Cts peptide (˜1.5 nM) decreases in patients with essential hypertension, the complex chronic disorder with a poorly understood pathogenesis, and also in normotensive subjects with a family history of hypertension and increased epinephrine secretion (12); this implicates that CST is an inhibitor of chromaffin cell catecholamine secretion in vivo. Genetic ablation of the CgA gene results in high blood pressure in mice which can be rescued either by pre-treatment with CST peptide or by the introduction of the human CgA gene in the CgA null background (13). Accordingly, decreased CST levels may predict augmented adrenergic responses to stressors and increased risk of hypertension.

To understand human genetic variation at the level of the CgA gene, O'Connor's group at UCSD systematically searched for polymorphisms at the CHGA locus, and reported 3 naturally occurring amino acid substitution variants within the region of CST (9). Although two of the Cts variants (Pro370Leu & Arg374Gln) were reported to be relatively rare (minor allele frequencies 0.3-0.6%, respectively), one variant, Gly364Ser (S352SMKLSFRARGYS364FRGPGPQL372; position in the mature CgA protein) showed an allele frequency of ˜3-4%. These variants displayed differential potencies toward inhibition of nicotinic cholinergic agonist-evoked catecholamine secretion from sympathochromaffin cells in vitro with the following rank order of potency Pro370Leu>wildtype (WT)>Gly364Ser>Arg374Gln (11). In vivo, human carriers of the 364Ser allele had profound alterations in autonomic activity, in both the parasympathetic and sympathetic branches, and may be protected against the future development of hypertension, especially evident in males (14). On the basis of this remarkable vasoreactivity of Cts, we reasoned that its human variants could also act as cardiotropic agents exerting differential effects on the heart.

The working heart and the arterial system are such close functional complements that the analysis of their dynamic interaction represents an obligatory step in the integral understanding of cardiovascular homeostasis both under normal and abnormal (hypertension) conditions. There have been no studies investigating the direct action of CST on isolated rat heart preparations, which being independent from extrinsic neuronal and endocrine influences is an ideal model for analyzing the direct cardiac effects of a substance. Using the Langendorff perfused rat heart as a mammalian cardiac paradigm, we show that CST and its naturally occurring human variants directly influence both inotropic and lusitropic functions. In addition to signaling analyses addressed to determine the possible mechanisms of action of WT-CST, we demonstrate that CST inhibits the positive inotropic actions of both isoproterenol (ISO) and endothelin-1 (ET-1). The results provide a novel insight into the role of CST as an endocrine/paracrine cardiac modulator and inhibitor of β-adrenergic and ET-1 actions on the heart, adding new aspects on the structure-function relationship of CST variants and their anti-hypertensive potential.

### MATERIALS AND METHODS

Animals—Male Wistar rats (Morini, Bologna, Italy S.P.A.) weighing 180-250 g were housed (three per cage) in a ventilated cage rack system under standard conditions. Animals had food and water access ad libitum. The investigation conforms to the Guide for the Care and Use of Laboratory Animals, according to NIH Publication No. 85-23, revised 1996.

Drugs—WT-Cts, the pro370leu variant (P370L-Cts) and gly364ser variant of Cts (G364S-Cts) were synthesized by the solid-phase method, using FMOC protection chemistry (8). Peptides were purified to >95% homogeneity by preparative reverse-phase HPLC (RP-HPLC) on C-18 silica colyhhtrgumns. Authenticity and purity of the peptides was further verified by analytical chromatography (RP-HPLC) and by electrospray-ionization or MALDI mass spectrometry. Isoproterenol hydrochloride (ISO), ET-1, the NO scavenger PTIO [2-phenyl-4,4,5,5-tetramethylimidazoline-1-oxyl-3-oxide], inhibitors of either nitric oxide synthase (N^{G}-monomethyl-L-arginine, L-NMMA) and (N5-(1-Imino-3-butenyl)-L-ornithine, L-VNIO) or guanylate cyclase (ODQ) or protein kinase G (KT5823) were purchased from Sigma Chemical Company (St. Louis, Mo., USA). All drug-containing solutions were freshly prepared before experimentation.

Isolated heart preparation—Rats were anesthetized with ethyl carbamate (2 g/Kg rat, i.p.), the hearts rapidly excised and then transferred in ice-cold buffered Krebs-Henseleit solution (KHs). As previously described (23), the aorta was immediately cannulated with a glass cannula and connected to Langendorff apparatus to start perfusion at constant flow-rate (12 ml/min). Briefly, the apex of the left ventricle (LV) was pierced to avoid fluid accumulation. A water-filled latex balloon, connected to a pressure transducer (BLPR, WRI, Inc. USA), was inserted through mitral valve into the LV allowing isovolumic contractions and continuous mechanical parameters recording. Another pressure transducer located just above the aorta recorded coronary pressure (CP). The perfusion solution consisted of a modified non re-circulating KHs containing (in mM) NaCl 113, KCl 4.7, NaHCO₃ 25, MgSO₄ 1.2, CaCl₂ 1.8, KH₂PO₄ 1.2, glucose 11, mannitol 1.1, Na-pyruvate 5 (pH 7.4; 37° C.; 95% O₂-5% CO₂). Hemodynamic parameters were assessed using a PowerLab data acquisition system and analysed using Chart software (both purchased by ADInstruments, Basile, Italy).

Basal conditions—Heart performance was evaluated from the LV pressure (LVP, in mmHg) which is an index of contractile activity, the rate-pressure product (RPP: HR×LVP, in 10⁴ mmHg×beats/min) which is an index of cardiac work (24), the maximal value of the first derivative of LVP (25) (mmHg/sec) which is an index of the maximal rate of LV contraction, the time to Peak Tension of isometric twitch (Ttp) which is an assessment of inotropism. Lusitropism was determined by calculating the maximal rate of LVP decline [−(LVdP/dT)max] (mmHg/sec), the half time relaxation (HTR) (sec), which is the time required for tension to fall from the peak to 50% and T/−t ratio obtained by +(LVdP/dT)max/−(LVdP/dT)max (26). Mean CP was calculated by averaging values obtained during several cardiac cycles (23).

Catestatin stimulated preparations—Repetitive exposure of each heart to a single concentration (33 nM) of WT-Cts revealed absence of desensitization (data not shown). Thus, concentration-response curves were generated by perfusing cardiac preparations with KHs supplemented with increasing concentrations of WT-Cts (from 11 to 200 nM) for 10 min.

Adrenergic and cholinergic receptors involvement—To obtain information on the involvement of β₁/β₂, α-adrenergic receptors (ARs) and cholinergic receptors (AchR) on the inotropic and lusitropic effects induced by WT-Cts, cardiac preparations, stabilized for 20 min with KHs, were perfused with Nadolol (10 nM) or Phentolamine (100 nM) or Atropine (10 nM) for 10 min and then washed-out with KHs. After returning to control conditions, each heart was perfused with KHs containing a single concentration of Cts WT (110 nM) plus Nadolol (10 nM) or Phentolamine (100 nM) or Atropine (10 nM) for an additional 10 min.

Involvement of inhibitory G-protein (Gi/o)—To evaluate the involvement of inhibitory G-proteins in the cardiac action of WT-Cts, hearts were preincubated for 60 min with KHs enriched with pertussis toxin (PTx: 0.01 nM) and then exposed for 10 min to WT-Cts (110 nM). PTx catalyzes ADP-ribosylation of Gi/o alpha-subunit, uncoupling Gi-membrane receptor interaction.

Cardiac signaling—Hearts were perfused with or without 110 nM WT-Cts as before. At the end of experiment hearts were snap frozen under liquid nitrogen and homogenized with 1 ml of ice cold 0.2 M sucrose, Tris maleate (pH 7.0) buffer supplemented with 2 mM EDTA, pH 8.0, 1 mM sodium orthovanadate, 10 mM sodium pyrophosphate, 1 mM phenylmethylsulfonyl fluoride, 10 μg/ml leupeptin, 10 μg/ml aprotinin and 0.1 mM 3-isobutyl-1-methylxanthine. SR membrane fractions were isolated from cytosolic proteins as described before (27). Protein content was determined by Bradford assay (BIO-RAD) and 100 μg of cytosolic protein was subjected to SDS-PAGE immunoblot analysis for phosphorylated-ERK, total ERK (P-ERK and T-ERK; Santa Cruz, Calif.). Two hundred μg cytosolic protein was used for immunoblot detection of phosphorylated-protein kinase B (Akt)-Ser473 (P-Akt), total-Akt (T-Akt), phosphorylated GSK-3 (P-GSK-3-Ser9) and actin (Cell signaling Technology, MA). For total phospholamban (T-PLN) and phosphorylated PLN (P-PLN-Ser16) level assessment, 10 μg of SR membranes were subjected to electrophoresis and the immunoblots were probed with anti-mouse PLN antibody (Affinity Bioreagents, CO) and anti-P-PLN-Ser16 (Badrilla, UK).

NO-pathway inhibitor stimulated preparations—Hearts were stabilized for 20 min with KHs, perfused with 110 nM WT-Cts for 10 min and then the peptide was washed-out with KHs. After returning to control conditions, each heart was perfused with KHs containing either the NO scavenger PTIO, the non-specific NOS inhibitor L-NMMA, a soluble guanylate cyclase inhibitor (ODQ) or a protein kinase G blocker (KT5823). Subsequently, the hearts were exposed to the specific signaling inhibitor plus 110 nM of WT-Cts.

Isoproterenol stimulated preparations—Cardiac preparations were stabilized for 20 min with KHs and then perfused with 5 nM ISO for 10 min. ISO was washed-out with KHs. After returning to control conditions, each heart was perfused with KHs containing a single concentration of WT-Cts (110 nM), P370L-Cts (33 nM) or G364S-Cts (200 nM) with 5 nM ISO for a further 10 min.

To further describe the antagonistic action of either WT-Cts (33 nM), P370L-Cts (110 nM) or G364S-Cts (200 nM) against ISO-dependent stimulation, dose-response curves were generated by perfusing the heart preparations with KHs enriched with increasing concentrations of ISO (0.1 nM to 1 μM) alone. These curves were then compared to those obtained by exposing other cardiac preparations to the same perfusion medium containing increasing concentrations of ISO (0.1 nM to 1 μM) plus a single concentration of either WT-Cts (33 nM) or P370L-Cts (110 nM) or G364S-Cts (200 nM).

ET-1 stimulated preparations—Hearts, stabilized for 20 min with KHs, were perfused for 10 min with ET-1 (1 nM) and then washed-out with KHs. After returning to control conditions, each heart was perfused with KHs containing a single concentration of either WT-Cts (33 nM), P370L-Cts (110 nM) or G364S-Cts (200 nM) plus 1 nM ET-1 for a further 10 min.

Statistics—Data are expressed as the mean±SEM. Curve fitting was accomplished in the program Kaleidagraph (Synergy Software, Reading, Pa.). Peptide EC₅₀ and IC₅₀ values were interpolated as the concentration that achieved 50% stimulation and inhibition, respectively. For analysis of phosphorylated proteins, levels were quantified using Bio-Rad QuantifyOne and volumes of phosphorylated proteins were divided by the levels of non-phosphorylated proteins to calculate fold activation. Stimulation of protein activity was expressed as fold increase over vehicle-treated control. The means and S.E.M. were calculated for each treatment. Multiple comparisons were made using either one-way ANOVA followed by Bonferroni's post-hoc test or two-way ANOVA. Statistical significance was concluded at p<0.05. Statistics were computed with the program InStat (GraphPad Software, Inc., San Diego, Calif.).

### RESULTS

Basal conditions—After 20 minutes of stabilization, the following basal recordings were measured: LVP=89±3 mmHg, heart rate=280±7 beats/min, RPP=2.5±0.1 10⁴ mmHg beats/min, CP=63±3 mmHg, +(LVdP/dT)max=2492±129 (mmHg/sec), T/−t=0.08±0.01 (sec), −(LVdP/dT)max=1663±70 (mmHg/sec), HTR=0.05±0.01 (sec) and T/−t or +(LVdP/dT)max/−LVdP/dT)max=1.49±1.84 (mmHg/sec). Endurance and stability of the preparation, analyzed by measuring performance variables every 10 min, showed that the heart preparation is stable for up to 180 min on the perfusion apparatus.

Inotropic and lusitropic actions of WT-Cts—To test whether WT-Cts alters basal cardiac parameters, heart preparations were exposed to increasing concentrations of WT-Cts to generate concentration-response curves. Exposure of single repeated doses of WT-Cts (33 nM) showed absence of desensitization (data not shown). WT-Cts effect on LVP reached its maximum at 5 min after administration, remaining stable for 15 min and then gradually decreased with time. Accordingly, cardiac parameters were measured at 10 min.

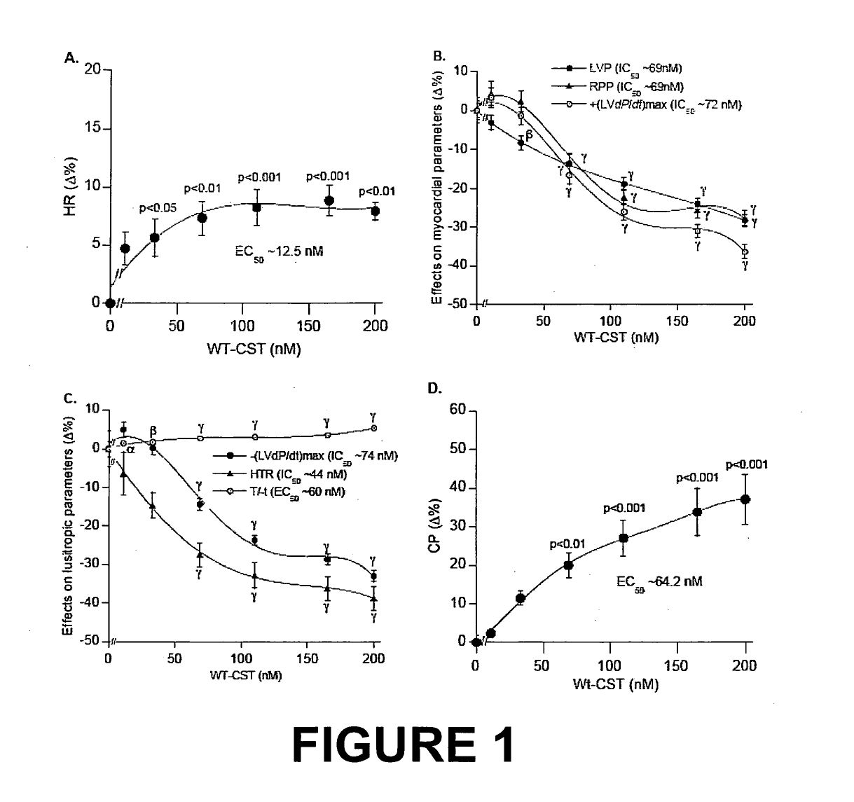

WT-Cts significantly increased FIR at 33 nM, reaching a maximum at 165 nM (EC₅₀˜12.5 nM) (FIG. 1A). Myocardial parameters were markedly inhibited by WT-CST: LVP (IC₅₀˜69 nM); RPP (IC₅₀˜69 nM) and +(LVdP/dt)max (IC₅₀˜72 nM) (FIG. 1B). Amongst the lusitropic parameters, WT-Cts caused a concentration-dependent increment in T/−t (EC₅₀₋₆₀ nM) and decrements in −(LVdP/dt)max (IC₅₀₋₇₄ nM) and HTR (IC₅₀˜44 nM) (FIG. 1C). WT-CST also caused a dose-dependent increment in CP with a maximum response at 200 nM (FIG. 1D).

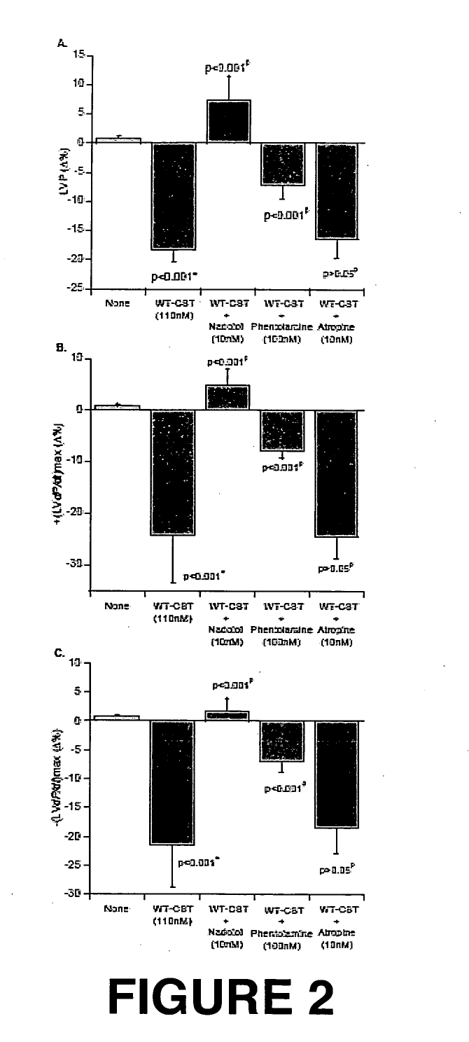

CST signaling to cardiac modulation—The negative inotropic and lusitropic effects of WT-CST were abolished by inhibition of β1/β2-ARs (by nadolol), reduced by α-ARs antagonist (phentolamine) or remained unaffected by inhibition of AChR (by atropine) (FIG. 2).

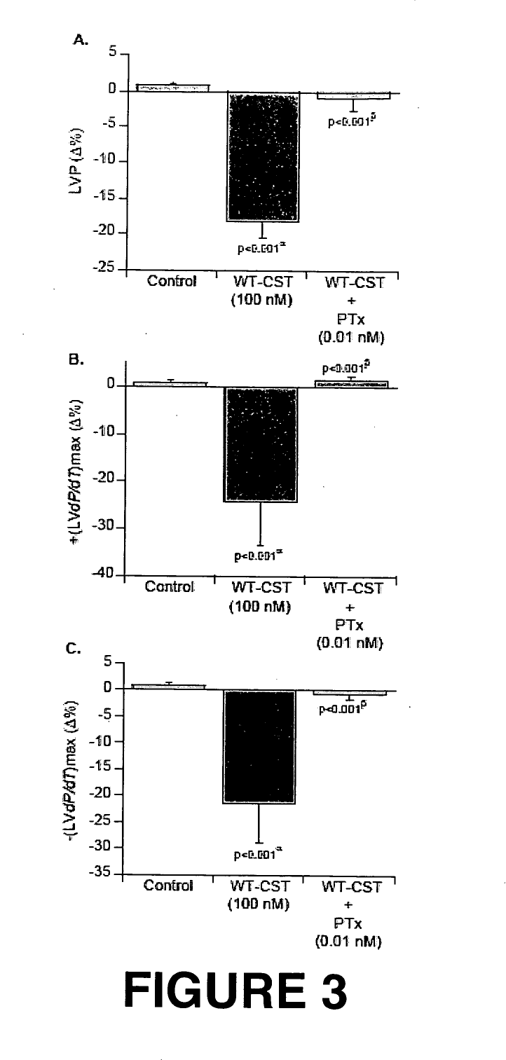

It is well established that Gi/o proteins are involved in the negative inotropism exerted by several cardiodepressive agents, including CgA-derived VS-1^{[24]}. To evaluate Gi/o proteins involvement in CST-dependent negative inotropism, hearts were perfused with the specific inhibitor PTx, in presence of Wt-CST (110 nM). PTx abolished Wt-CST-mediated negative inotropism (LVP and +(LVdP/dT)max), and lusitropism −(LVdP/dT)max), indicating Gᵢ involvement in CST signaling (FIG. 3).

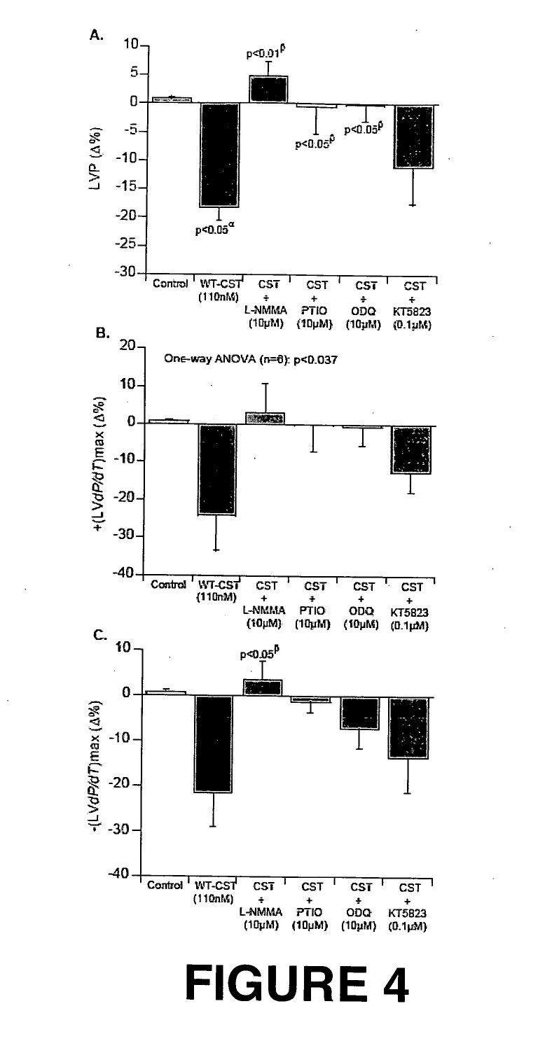

The NOS-NO-cGMP-PKG cascade plays a key role in the control of contractile performance in mammals (28). Accordingly, we tested NO-cGMP-PKG involvement in WT-CST-dependent cardiotropism by perfusing heart preparations with either PTIO (10 μM), L-NMMA (10 μM), ODQ (10 μM) or KT5823 (0.1 μM) in presence of CST. Antagonist concentrations were selected from preliminary dose-response curves that determined the minimum antagonist concentration that did not alter basal cardiac function. WT-CST (110 nM)-induced reduction of negative inotropism (i.e, +(LVdP/dT)max), and lusitropism (i.e., −(LVdP/dT)max) was abolished by NO removal by PTIO, NOS inhibition by L-NMMA and sGC blockade with ODQ. PKG inhibition by KT5823 failed to affect CST-induced changes in cardiac performance (FIG. 4). In addition, preliminary experiments performed with selective NOS inhibitors have shown that the LVP reduction induced by WT-Cts (LVP=−18.19±2.27) was abolished by L-NIO, eNOS selective inhibitor, (LVP=5.04±1.69) and reduced by Vinyl-L-NIO, nNOS selective inhibitor (LVP=−13.14±2.25). This indicates that Cts signals specifically through eNOS.

The mechanism of action of Cts is summarized in Table 1.

TABLE 1
Signaling pathways involved in the negative inotropic action of WT-Cts.
Inotropism was evaluated in the presence of selective antagonists of
α- and β-adrenergic, muscarinic receptors, Gi/o proteins and
the NO pathway.

| | |
|---|---|
| Signaling pathways | Negative inotropism |
| α-adrenoceptors | Reduced |
| β-adrenoceptors | Abolished |
| Muscarinic receptors | Unchanged |
| Gi/o proteins | Abolished |
| eNOS-NO-cGMP-PKG | Abolished |

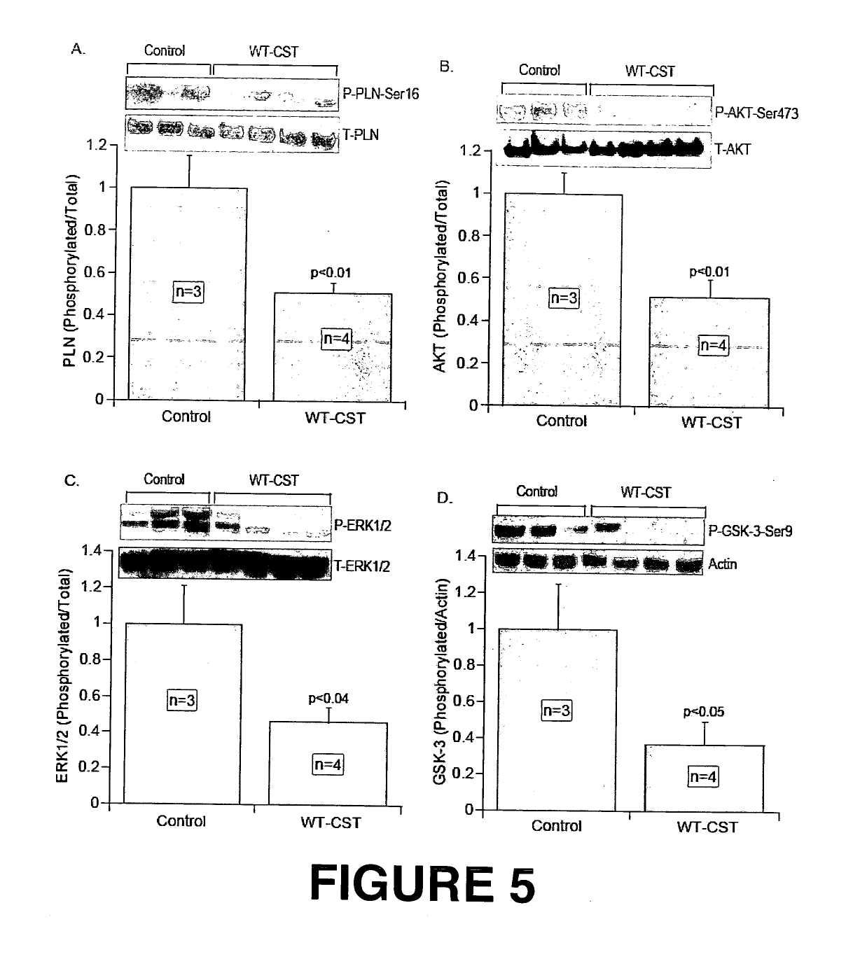

Intracellular Ca²⁺ coordinates cardiac contraction and relaxation. PLN, i.e. the 52-aminoacid transmembrane SR phosphoprotein regulating Ca²⁺ ATPase SERCA2a, in its dephosphorylated state inhibits Ca²⁺ pump activity. PLN phosphorylation alters the PLN-SERCA2a interaction, relieving Ca²⁺-pump inhibition and enhancing relaxation rates and contractility. Phosphorylation of Protein Kinase B (Akt), GSK-3 (glycogen synthase kinase-3) and ERK1/2 (extracellular signal regulated kinase), proteins also modulate cardiac function. Therefore, we checked whether WT-Cts signals through these proteins. WT-Cts inhibited phosphorylation of PLN at the PKA specific site, Ser16 (FIG. 5A), AKT at Ser473 (FIG. 5B), ERK1/2 (FIG. 5C) and GSK-3β at Ser9 (FIG. 5D)

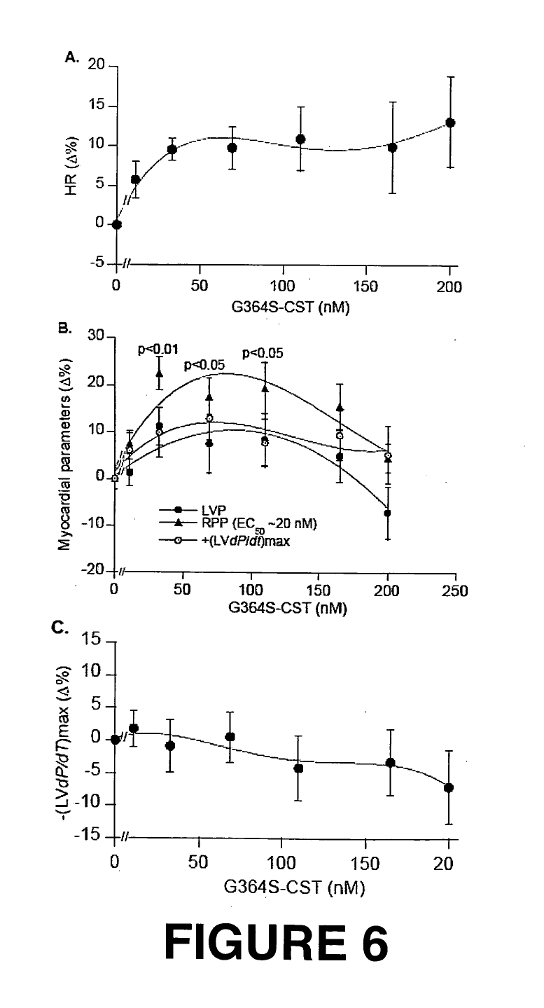

Inotropic and lusitropic actions of naturally occurring human CST variants—G364S-CST caused a significant increase in RPP even at 33 nM (FIG. 6B). Other parameters tested were unaffected by treatment with this variant (FIG. 6A-C).

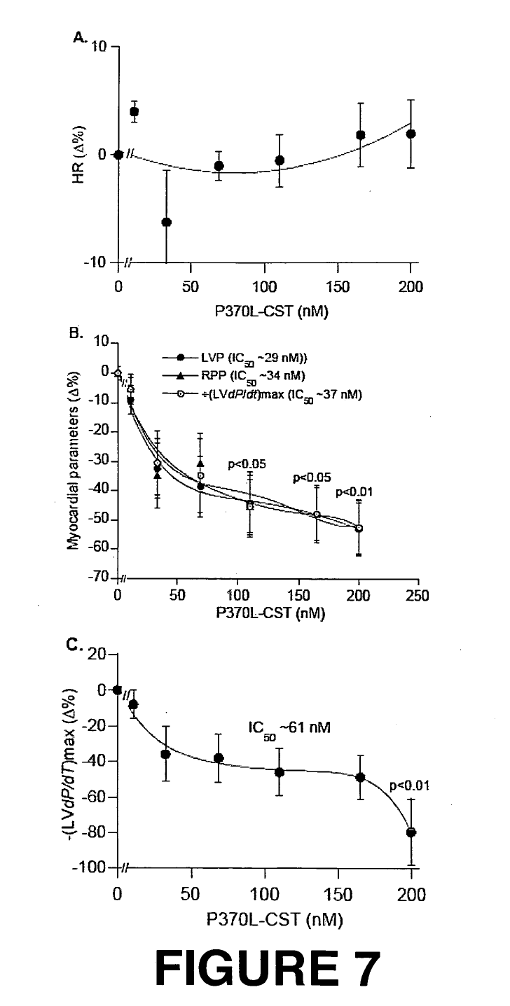

P370L-CST induced a negative inotropism from 110 to 200 nM, the maximum decrease in LVP and RPP being at concentration of 200 nM (FIG. 7B). This peptide also caused marked decrease in +(LVdP/dT)max starting from 110 nM, with a maximum decrease at 200 nM (FIG. 7B) and −(LVdP/dT)max (FIG. 7C). These effects were not accompanied by changes in HR (FIG. 7A) and CP (data not shown).

Inotropic and lusitropic effects of the three CST variants are summarized in Table 2.

TABLE 2
Effects of WT-CST, P370L-CST and G364S-CST on cardiac parameters
under basal conditions.

| Cardiac | | | |
|---|---|---|---|
| parameters | WT-CST | P370L-CST | G364S-CST |
| Inotropism | Negative | Negative | No effect |
| Lusitropism | Negative | No effect | No effect |

Activity: WT-CST > P370L-CST > G364S-CST

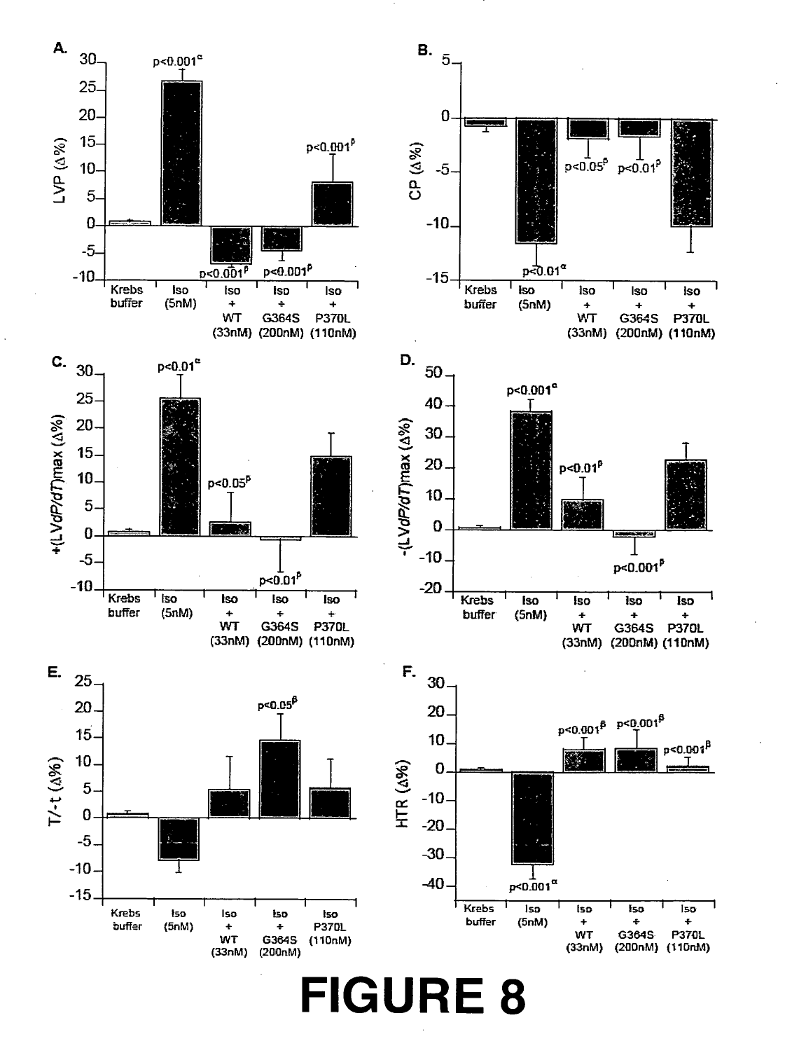

CST modulation of isoproterenol-induced cardiac changes—Cardiac preparations exposed to the β-adrenergic agonist ISO (5 nM) revealed positive inotropic and lusitropic responses that were associated with vasodilation. These modifications were indicated by an increase in LVP, RPP, +(LVdP/dT)max, −(LVdP/dT)max, HTR, T/−t and a decrease in the CP, (FIG. 8). As reported previously (29, 30), ISO effects were significant up to 5 min from its initial application. Hearts were perfused with KHs containing ISO (5 nM) plus a single concentration of either WT-CST (11 nM, 33 nM, or 110 nM), P370L-CST (110 nM) or G364S-CST (200 nM). All parameters were measured at 5 min after the drug application. All three peptides abolished the ISO stimulatory effect on LVP in the following rank order: WT-CST>G364S-CST>P370L-CST (FIG. 8A). The ISO induced coronary dilation was blocked by both WT-CST and G364S-CST, remaining unchanged by P370L-CST (FIG. 8B). In addition, CST peptides counteracted the ISO-dependent positive inotropism. In fact, WT-CST (by ˜90%) and G364S-Cts (by >100%) blocked +(LVdP/dT)max (FIG. 8C). Likewise, the ISO-induced positive lusitropism was blocked by WT-CST and G364S-CST. P370L-CST failed to modulate the lusitropic effect of ISO (FIG. 8D). The ISO-dependent decrease of T/−t was blocked only by G364S-CST (FIG. 8E). All three peptides blocked ISO-induced HTR (FIG. 8F).

To characterize the inhibitory action of either WT-CST (11, 33 or 110 nM), G364S-CST (200 nM) or P370L-CST (110 nM) toward ISO-dependent stimulation of cardiac function, heart preparations were perfused with KHs containing increasing concentrations of ISO (0.1 nM to 1 μM) either alone or in combination with one of the three CST variants.

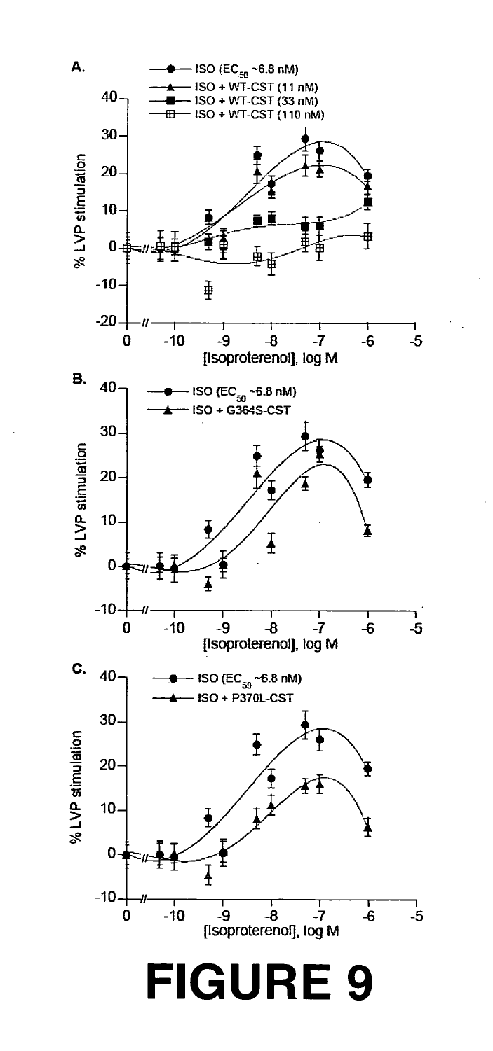

ISO alone increased LVP significantly from at concentrations ranging from 5 nM to 1 μM (FIG. 9). The EC₅₀ values of the % increase in LVP stimulation were determined from increasing concentrations of ISO alone or ISO plus either WT-CST (11, 33 and 110 nM), P370L-CST (110 nM) or G364S-CST (200 nM). WT-CST at concentrations of 33 and 110 nM, G364S-CST at a concentration of 200 nM and P370L-CST at a concentration of 165 nM significantly inhibited ISO-induced stimulation of LVP and displayed an interaction with ISO (FIG. 9). The EC₅₀ values of LVP (in log M) of ISO alone were −8.67±0.3 (r²=0.84), of ISO plus WT-CST at concentrations of 11, 33 and 110 nM were −8.7±0.35 (r²=0.85), −7.35±0.46 (r²=0.73) and −7.2±1.14 (r²=0.29), respectively. ISO plus P370L-CST (165 nM) resulted in an EC₅₀ value of LVP at −8.51±0.46 (r²=0.76). Since increasing concentrations of ISO failed to overcome the antagonistic effect of CST, the actions of CST are considered as a non-competitive type of antagonism (FIG. 9). The counteraction of adrenergic stimulation by CST is summarized in Table 3.

TABLE 3
Anti-adrenergic actions of CST and its variants

| Cardiac | | ISO + WT- | ISO + | ISO + |
|---|---|---|---|---|
| effects | ISO alone | CST | P370L-CST | G364S-CST |
| Inotropism | Positive | Abolished | Abolished | Abolished |
| Lusitropism | Positive | Abolished | Abolished | Abolished |
| Coronary | Vasodilation | Abolished | No effect | Abolished |
| pressure | | | | |
| EC₅₀ | 8.67 ± 0.3 | 7.2 ± 1.14 | 8.51 ± 0.46 | 8.61 ± 0.57 |

Order of potency: WT-CST > G364S-CST > P370L-CST

CST blockade of ET-1 stimulated cardiac functions—To verify Cts's ability to counteract the ET-1-mediated inotropic and coronary effects, hearts were perfused with KHs containing ET-1 alone or in combination with one of the Cts variants.

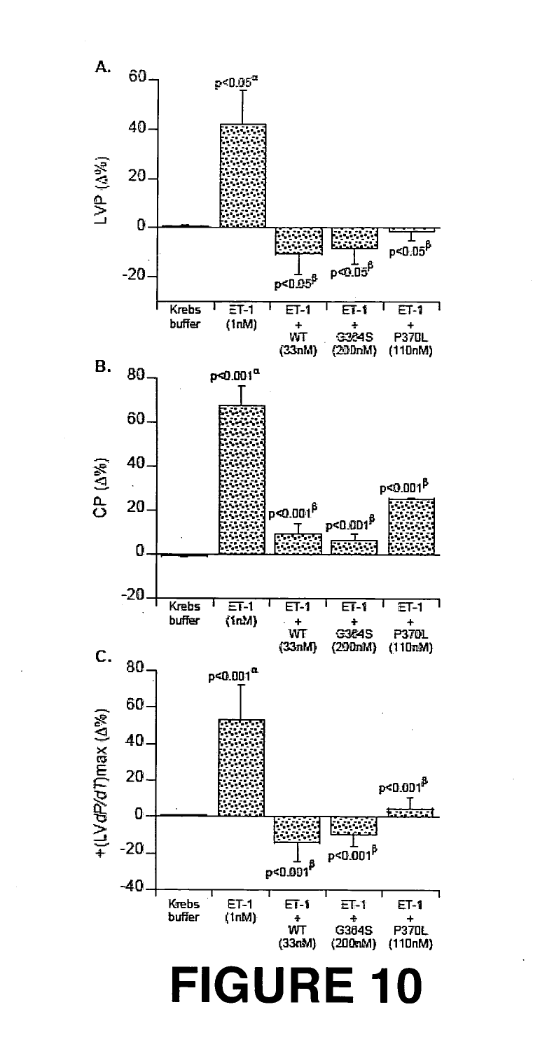

Administration of ET-1 alone induced a dose-dependent biphasic effect on contractility. At a concentration of 1 nM ET-1 increased LVP and +(LVdP/dT)max, whilst at higher doses (10 nM) CST decreased both parameters, thus inducing a negative inotropic effect. CST peptides differentially affected the ET-1 induced inotropic effects. In fact, positive inotropism was blocked by WT-CST (33 nM), P370L-CST (110 nM) and G364S-CST (200 nM) (FIG. 8). Moreover, ET-1 alone (1 and 10 nM) typically induced a significant increase in coronary constriction (31), which was blocked by all three human CST variants (FIG. 10).

### CONCLUSION

In conclusion, since the heart and the arterial system interact as a closed-loop control system, the former being a target organ of prolonged and excessive adrenosympathogenic activity, such as hypertension with consequent hypertrophy and ischemic cardiomyopathy, it is of relevance that the recently discovered anti-hypertensive modulatory action of CST now appears associated with a direct powerful counter-excitatory cardiac action. This cardiotropism of CST may be inherently important for normal heart function, but also suggests a unique inhibitory role of the peptide under abnormal cardiac conditions characterized by adrenosympathogenic over-activation. Until now, blockade of the β-adrenergic receptors is one of the most effective pharmacologic interventions in hypertensive patients. Future studies of administration of CST to hypertensive animal models will determine whether the peptide has also any therapeutic potential for the treatment of hypertensive cardiomyopathy.

### EXAMPLE 2

### Catestatin Acts as an Anti-Stress Peptide and Also Restores Baroreflex Sensitivity in Hypertensive Mice

### MATERIALS AND METHODS

Animals. Chga knockout (KO) mice and their wild-type (WT) littermates were generated as described (13). All animals were housed on a 12 hr light dark cycle and fed a standard rodent chow. The Animal Care and Use Committee of University of California at San Diego approved all protocols for animal use and euthanasia in accordance with National Institute of Health guidelines. Both WT and KO mice (5-6 month old) in this study were from mixed (129 SvJ and C57BL/6) genetic background.

Conscious mice. Measurement of BP and HR in immobilization stress-induced telemetered mice: BP & HR were measured by telemetry as described previously (13). The experiments were initiated 10 days after the surgery. Immobilization stress was initiated by placing mice in restrainer (Braintree Scientific Inc, Braintree, Mass.) at 10:00 AM and kept for 2 hrs for continuous recording of BP and HR by telemetry followed by recovery from stress in home cages for 2 hrs. CST was injected (40 μg/g bw ip) 30 minutes before stress induction to explore the role of CST in stress response.

Catecholamine assay following immobilization stress: Mice were anesthetized by inhalation of “Isoflurane, USP” and blood was collected from the left ventricle in potassium EDTA tubes. Isoflurane anesthesia was found to be best for measurement of plasma catecholamines with minimal stress-induced variation. Plasma catecholamine was measured by HPLC connected to an electrochemical detector (Waters 600E Multisolvent Delivery system and Waters 2465 Electrochemical Detector, MA) in a group of restraint mice (non-telemetered). Separation was performed on an Atlantis dC18 column (2.1×150 mm, 3 μm) from Waters. The mobile phase (0.15 ml/min) consisted of phosphate-citrate buffer and 2.2% N, N-Dimethylacetamide and acetonitrile at 95:5 (V/V). An internal standard 3,4-Dihydroxybenzylamine (DHBA, 1 ng) and an antioxidant sodium meta-bisulfite (final concentrations of 0.125 mM) were added to 0.25 ml of plasma. Following the addition of 15 mg of alumina (Aluminium oxide, Activity Grade: Super I, Type WA-4, Sigma-Aldrich, PA), the pH of the solution was raised to pH 8.6 by adding Tris buffer (0.96 M Tris, 50 mM EDTA, pH 8.69). Following a 30 min incubation and centrifugation (5000 rpm, 5 min), the supernatant was discarded and the beads were washed with water. The catecholamine was eluted with 80 ml of 0.1 N HCl supplemented with 0.1 mM sodium meta-bisulfite. The data were analyzed using Empower software. Catecholamine levels were normalized with the recovery of internal standard.

Unconscious mice. Surgical procedures for hemodynamic measurements: Hemodynamic evaluation in both WT and KO mice was performed under general anesthesia [ketamine (100 mg/kg) and xylazine (2.5 mg/kg)] while connected to a ventilator. LV pressure was monitored by Millar micromanometer catheter of size 1.4 French (0.46 mm; Millar Instruments, Houston, Tex.); it was inserted into the LV where phasic and mean pressure was continuously monitored (Gould, Cleveland, Ohio). A venous catheter (MicroRenathane tube MRE-033; Braintree Laboratories, Braintree, Mass.) is implanted on right femoral vein for injection of peptide and other reagents. All physiologic signals were acquired by WINDAQ (Dataq Instruments, Akron, Ohio) and analyzed by BP-Analysis software (developed for own laboratory).

Assessment of baroreflex sensitivity (BRS): BRS (in milliseconds per mmHg) was assessed by both high-pressure [phenylephrine (PE) bolus] and low-pressure [sodium nitroprusside (SNP) bolus] stimuli in unconscious mice. Although conscious animals provide optimal physiological data, a host of undesirable signals are triggered by physical activities, and sleep cycle alterations in behavioral state. Therefore, we have chosen to conduct the experiments in unconscious mice to avoid some of the pitfalls encountered in awake animals. The absolute changes of HR in response to changes in SBP induced by PE or SNP were subjected to linear regression analysis to determine BRS. To test the effect of CST on BRS in KO mice, CST was injected (40 μg/g bw iv) 30 min before the injection of PE or SNP through the catheter implanted into the femoral vein.

BRS to high-pressure stimulus. High pressure BRS was evaluated by recording diminution of HR in response to PE-induced hypertension. Changes in SBP and HR were recorded continuously for 0.5 min after a bolus injection of PE (0.005 μg/g bw iv). This dose was selected from a preliminary dose-response study (0.001-0.05 μg/g bw iv).

BRS to low-pressure stimulus. This was evaluated by recording increments in HR in response to SNP-induced fall in BP. Changes in SBP and HR were recorded continuously for 0.5 min after a bolus injection of SNP (0.05 μg/g bw iv). This dose was selected from a preliminary dose-response study (0.01-0.1 μg/g bw iv).

Statistical analysis. Statistical analyses were done as described in Example 1.

### RESULTS

### I. Physiology: Stress Responses in Conscious Mice.

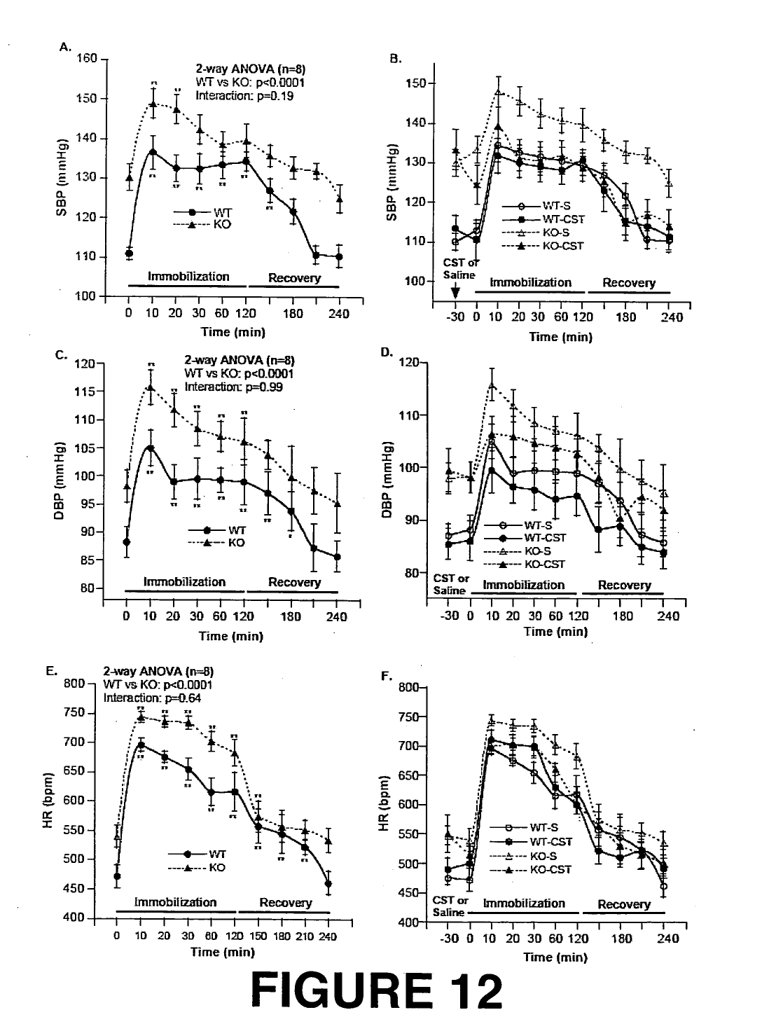

SBP. SBP increased dramatically 10 min after immobilization stress in both WT (by 25.5 mmHg) and KO (by 18.6 mmHg) mice (FIG. 12A). In WT mice, the BP remained high during the 2 hr of immobilization stress; in KO mice, BP returned to normal level 30 min after stress. SBP returned to normal level after 1 hr of recovery in WT mice. The immobilization effect on SBP in KO mice was significantly higher compared to WT (AUC: WT, 30387±417 versus KO, 32852±405; p<0.001).

In WT mice, CST had no effect on basal SBP; CST attenuated (by 7.1 mmHg) stress-induced increments in SBP (FIG. 12B). In contrast, in KO mice, CST decreased basal SBP, attenuated stress-induced increments in SBP and brought SBP to WT level after 1 h of recovery from the stress (FIG. 12B). Overall, CST caused significant attenuation of stress-induced elevation of SBP in KO mice only as compared to saline-treated group (AUC: KO-S, 29092±425 versus KO-CST, 26639±399; p<0.0009).

DBP. KO mice displayed significantly higher basal DBP compared to WT mice (98.2±2.9 vs. 88.2±2.7 mmHg; p<0.025) (FIG. 12C). DBP increased both in WT (by 16.8 mmHg) and KO (by 17.6 mmHg) mice after 10 min of stress (FIG. 12C). In both the groups, DBP remained high during 2 hr of stress and returned to normal level 60 min (KO) or 90 min (WT) of recovery. The stress-induced increase in DBP is higher in KO mice compared to WT (AUC: WT, 23018±305 versus KO, 24998±523; p<0.006).

In both WT and KO mice, CST had no significant effect either on basal or stress-induced increments in DBP as compared to saline-treated group (FIG. 12D).

HR. Resting HR was significantly higher in KO mice compared to WT mice (FIG. 12E). Stress-induced increase in HR after 10 min of stress was comparable in WT (by 224 bpm) and KO (by 204 bpm) mice. In KO mice, HR remained high during the stress period (FIG. 12E) and returned to basal level after 60 min. Overall, stress-induced changes on HR in KO mice were significantly higher compared to WT (AUC: WT, 140375±3098 versus KO, 153191±2101; p<0.005)

In WT mice, CST had no effect on the basal and stress-induced increase in HR (FIG. 12F); in KO mice, CST abolished heightened HR responses to stress as compared to saline-treated group (AUC: KO-S, 153145±2153 versus 143340±1889; p<0.004).

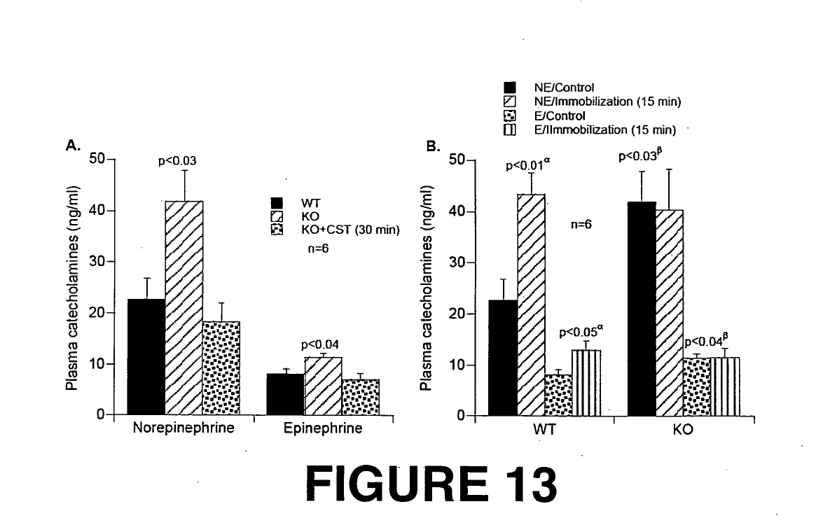

II. Biochemistry: Catecholamines in conscious mice. As reported previously (13), KO mice showed higher basal plasma norepinephrine (by 85%) and epinephrine (by 42%) (FIG. 13A), that returned to WT level upon CST replacement (FIG. 13A). In WT mice, immobilization stress (15 min) increased both plasma norepinephrine (by 92%) and epinephrine (by 61%); in KO mice, stress had no effect on plasma catecholamine (FIG. 13B).

### Pharmacology: Response to Exogenous Pressor/Depressor Agents in Unconscious (Anesthetized) Mice

Effects of Intravenous PE or SNP for 0.5 Min.

SBP. In KO mice, anesthesia completely abolished heightened SBP (FIG. 3A) and HR (FIG. 3B). PE (0.005 μg/g bw) significantly increased SBP both in WT (by 34.1 mmHg) and KO (by 45.6 mmHg) mice (FIG. 4A; WT versus KO: p<0.014). SNP (0.05 μg/g bw iv) caused significant decrease in SBP in both the WT (by 40.1 mmHg) and KO (by 61.6 mmHg) mice (FIG. 4B; WT versus KO: p<0.0005).

HR. In WT mice, PE (0.005 μg/g bw) caused significant decrease in HR (by 26 bpm) (FIG. 4C) as a result of reflex bradycardia. The HR changes were significantly different between WT and KO mice (p<0.005). In contrast, in KO mice, PE-induced bradycardia was completely abolished (FIG. 4C), possibly indicating impairment in vagal tone. As expected, SNP (0.05 μg/g bw) increased HR in both the WT (by 19.9 bpm) and KO (by 13.9 bpm) mice (FIG. 4D). Reflex tachycardia in response to SNP was not significantly different between WT and KO mice.

### Baroreceptor Function.

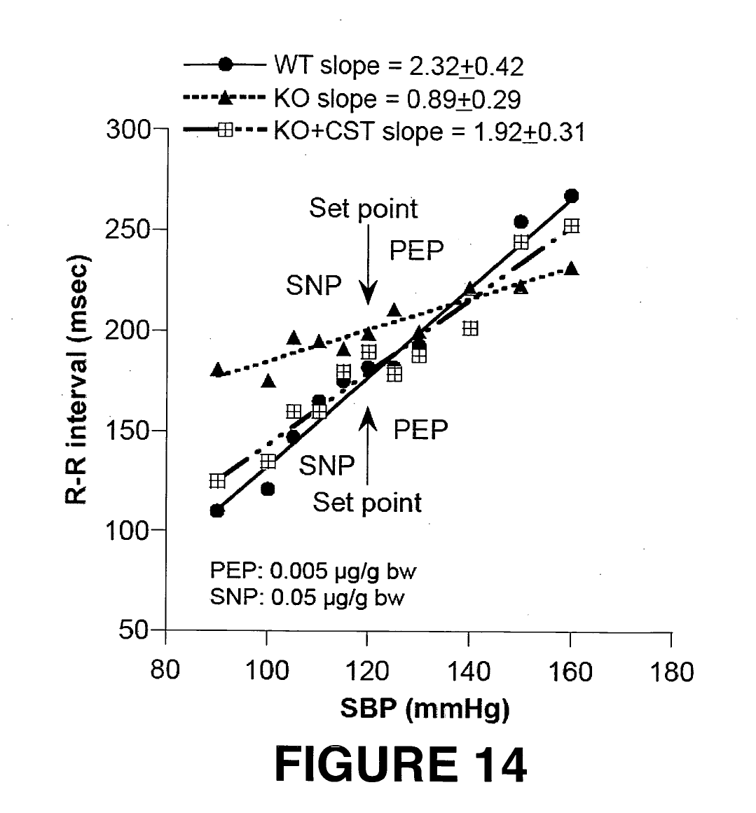

Baroreflex slope. Time-dependent cumulative effects of PE (0.005 μg/g bw) and SNP (0.05 μg/g bw iv) are shown in FIG. 14. Consistent with hypertensive subjects, the baroreflex slope in KO mice was decreased by ˜3-fold (WT: 2.32±0.42 vs. KO: 0.89±0.29; p<0.02) in response to PE (0.005 μg/g bw) and SNP (0.05 μg/g bw) (FIG. 5). In addition, the set point was increased in KO mice. CST supplementation restored dampened baroreflex slope in KO mice (FIG. 14).

### CONCLUSION

Genetic variation at the human CHGA locus has profound consequences for control of BP, and the Chga null mouse displayed profound hypertension and increased catecholamine secretion. We sought to understand such changes, and probed autonomic function in this model by physiological, biochemical, and pharmacological means. Our results suggest global disruption of autonomic function in this model, both parasympathetic and sympathetic. Defects in baroreceptor function may underlie the cardiovascular instability and exaggerated sympathetic outflow observed in KO animals. At least some of these abnormalities can be “rescued” by administration of the CHGA catecholamine release-inhibitory fragment CST. Since the CgA and CST mechanisms are altered in human hypertension, our results in this experimental animal model may provide insight into the pathogenesis of this common disorder.

### EXAMPLE 3

### CST Widens Heart Rate Variability (HRV) in Hypertensive Mice

### BACKGROUND

Cardiovascular performance is controlled by the autonomic nervous system (ANS). Beat-to-beat fluctuation in the heart rate (HR) is a balanced consequence of ANS tone to the heart, both sympathetic (increasing HR) and parasympathetic (decreasing HR). A decrease in heart rate variability (HRV) is longitudinally predictive of increased cardiovascular morbidity and mortality. Thus, we examined whether both short- and long-term HRV concerning temporal and spectral features recorded from the sedated mice to identify perturbations of the autonomic system in the chromogranin A knockout (CgA-KO) mice, which has earlier been reported to have echocardiographic abnormalities and prone to hypertension. Use of CST ameliorates both systolic and diastolic BP elevations (13). Since ANS status can be reliably monitored by analyzing time and frequency-domain characteristics of HRV, we sought to use the technology to evaluate functional alterations in ANS activity of CgA-KO mice.

### MATERIALS AND METHODS

Animals. Six Wild-type and seven CgA-KO mice were used in this study (see example 1 for the details).

Mice were sedated by an intraperitoneal injection of a 1 μl solution per gm body weight, containing Ketamine (100 mg/ml)+Xylazine (20 mg/ml) and Acepromazine (10 mg/ml). Mice were gently strapped onto the surgical table in a fixed supine position with silk tape. Body temperature was continuously monitored on an analog thermometer, model 44TA (YSI, Ohio) using a rectal probe, and maintained between 36.5 and 38.5° C. throughout the experiment using a controlled heating box. The analog output signal fed into the chart recorder (AD Instruments, Colorado) and suitably amplified. The adequacy of sedation was regularly verified by absence of vibrissae twitching and withdrawal reflex to a noxious toe-pinch and was supplemented intraperitoneally as needed. Animals breathed ambient air spontaneously throughout the experimental run. Investigational agent, a nicotinic cholinergic receptor antagonist (8), CST was dissolved in distilled water and injected intraperitoneally at a volume of 70 μl from a 1 mM working solution.

EKG electrodes were attached to all four limbs (incl. reference electrode) via 30G stainless steel hypodermic needles (Popper & Sons, New York) so as to record standard bipolar Einthoven derivation (lead I, II and III) EKG signals. The EKG signals were bandpass-filtered between 0.1 and 3 kHz with a notch filter set at 60 Hz, and sampled at 2 kHz. The voltage output fed into a data acquisition system (PowerLab-8, AD Instruments) for eventual off-line measurements and analysis by chart and scope (Labchart 6) software. Heart rate and rhythm were also monitored continuously on an oscilloscope (Tektronix 5113, Oregon) throughout the experiment.

EKG traces were edited using the Chart® software package. High frequency noise from the EKG was removed using a 45 Hz low-pass filter to enable detection of the R-waves, whenever appropriate. A threshold electrical potential value was set for each trace and the post-threshold maximum was taken as the R-wave. Each trace was visually inspected for any undetected R-waves and these were manually inserted, while incorrectly detected R-waves or non-normal beats such as extrasystoles were deleted. Time and frequency domain analysis of HRV was performed using fast Fourier-transformation with a Hanning window. Spectral power was calculated on 50% overlapped blocks on sequences of 1024 points (beats). Time-domain parameters recorded or calculated included: mean R-R interval (in ms) [i.e., NN interval, i.e., normal-to-normal intervals]; standard deviation of all normal R-R interval (SDNN, in ms), reflecting total autonomic variability; square root mean of squared differences between adjacent normal R-R intervals (RMSSD), an index for short-term variations in inter-beat intervals, primarily of parasympathetic nature; and percentage of normal consecutive inter-beat intervals differing by >7 ms (NN7%), reflecting cardiac parasympathetic tone, were calculated directly from the sequence of interval times. NN7 is a murine-adjusted value based on PNN50 determined for humans (32). The mean HR was calculated as the mean sequence of reciprocals of the interval times. For non-linear measurements on the return map of R-R intervals (Lorenz plot), the images acquired by the chart software were imported into Image J (National Institutes of Health, Bethesda) for evaluating point dispersion of S1 and S2 (see below). The width and length of the dispersion of data in Lorenz plots reflects the level of short- and long-term beat-to-beat variability, respectively. The different frequency domain measures of HRV were computed using cut-off frequencies for power in the very low frequency (VLF), low-frequency range (LF) and high-frequency range (HF) based on human studies multiplied by a factor of ten for HR adjustment (the approximate ratio between murine and human HR) (33). The area under the curve was calculated for the very-low-frequency (VLF: up to 0.4 Hz), low-frequency (LF: 0.4 to 1.5 Hz), and high frequency (HF: 1.5 to 4.0 Hz) bands, as previously defined in the mouse species (34). Total power (variance of normal R-R intervals over temporal segment) was defined as DC 4 Hz. Spectral variability at LF and HF bandwidths was also normalized to the total spectral area. Five-minute epochs of heart rate as a function of time were used for power spectrum (power) analysis. In the present paper we report the area of the LF band in milliseconds squared, the area of the HF band in milliseconds squared and the total power between 0.0033 and 4.0 Hz (total power) in milliseconds squared. Variables not normally distributed were naturally log-transformed. In order to investigate if HF portion of the HRV is coupled to respiration, we recorded an electromyogram (EMG_{di}) in two mice, using fine Teflon-coated silver wire with bare tip hooked onto the costal diaphragm following a laparotomy, and output signal suitably amplified and stored for analysis.

Mice with atrial fibrillation, excessive ectopic beats or technically inadequate recordings were excluded.

Statistical analyses. Statistical analyses were carried out as described in Example 1.

### RESULTS

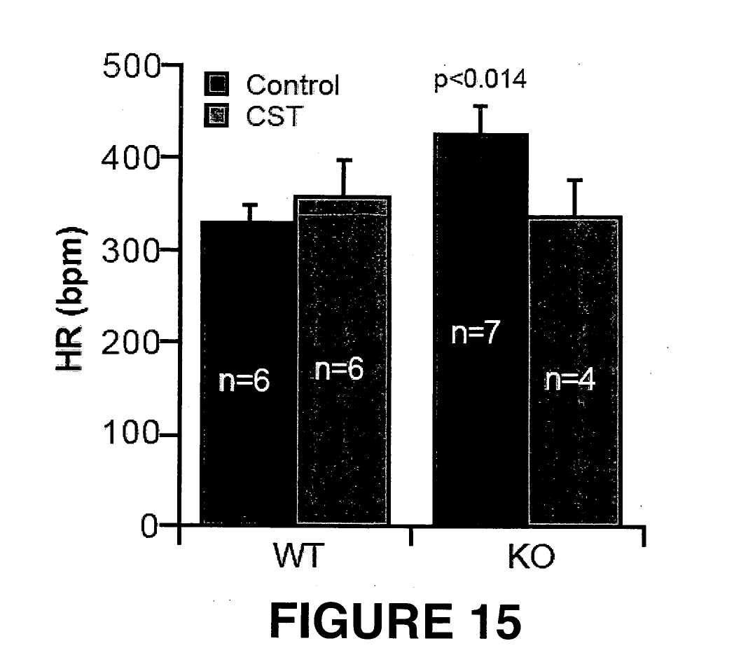

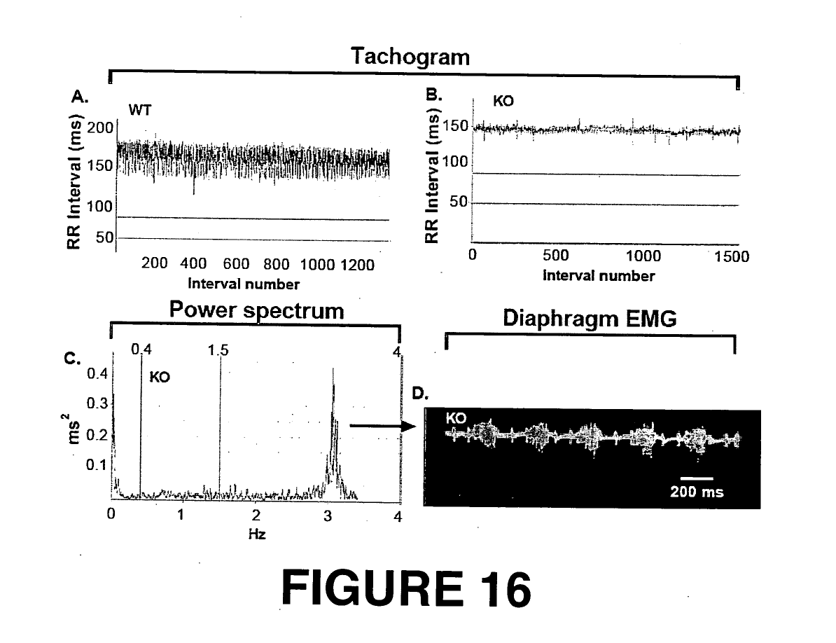

Frequency domain, and effects of catestatin. At baseline, KO mice showed higher heart rate compared with the wild-type group (427±31 vs. 330±20 beats.min⁻¹, p=0.014) (FIG. 15). In the heart rate tachogram of WT, the fluctuation width is evidently bigger than that of KO group and both animal groups appear to have spectral peaks at 2.5-4 Hz band, which seems to coincide with the central respiratory output, as reflected by the diaphragmatic electromyogram. FIG. 16 shows cardiac rate tachogram and corresponding power spectra for KO and WT mice.

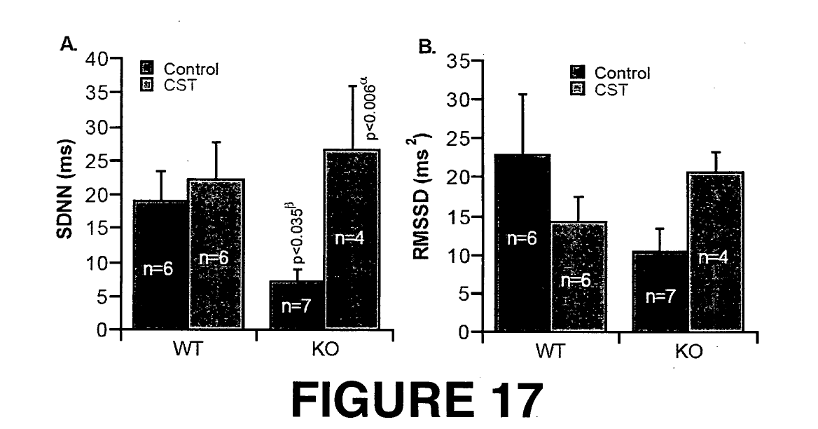

Time domain. As shown in FIG. 17, the standard deviation of beat-to-beat variation (SDNN) in KO mice was significantly lower compared with the values obtained from the wild-type group in the pre-catestatin stage. It also revealed that all time-domain parameters of HRV (SDNN, NN7%, RMSSD) were markedly lower in KO mice, and could be reversed by the use of catestatin.

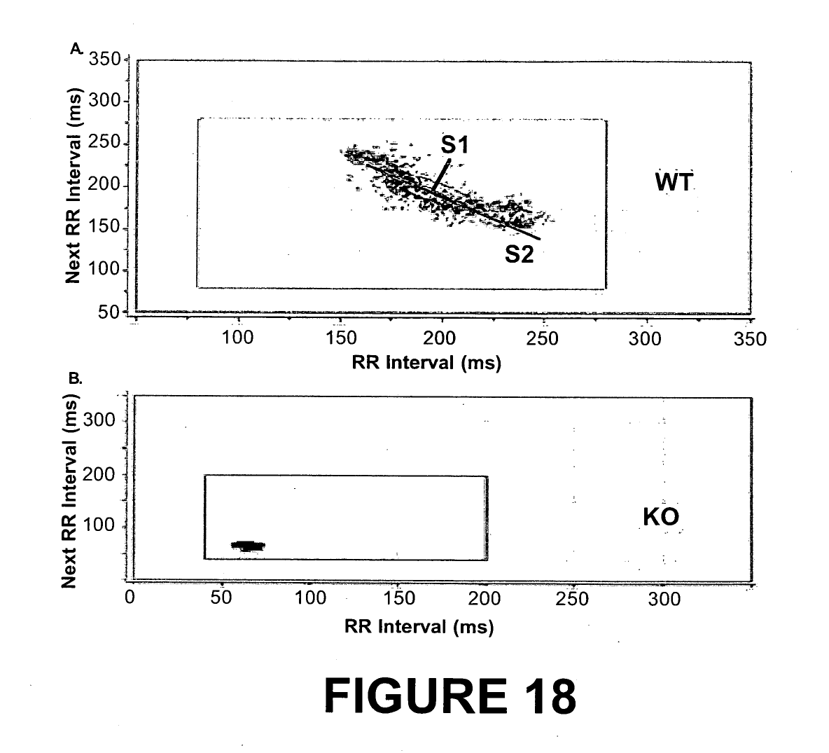

Lorenz analysis. The reductions in time-domain parameters were further supported by the non-linear measurements of R-R intervals, that showed parallel decrease in the index of short-term variability (S1: axis perpendicular to the line of identity) and that of S2 axis (along the line of identity) that reflects short-to-long term variability, primarily of sympathetic nature. These values are plotted in FIG. 18. As in the case of time domain parameters, intervention with catestatin caused marked improvements in the short-term and long-term variability of HRV.

Fourier transform to the frequency domain. Fourier transform power spectral analysis of HRV in WT and KO mice in the resting state showed periodic components of HRV in 2 distinct peaks mostly centered around 2.4-3.2 Hz (HF), considered to index cardio-vagal activity and below 0.2 Hz (VLF) in most instances, with occasional peak at 1 Hz. Spectral peak positions were not visibly altered under the influence of CST treatment. At baseline, KO mice had reduced VLF power. Treatment with CST did not have much influence on the HRV VLF parameters (Control: 2.156±0.939; CST: 2.468±0.69 [ln ms²]) in WT mice. However, in KO mice, CST increased HRV in the VLF band (Control: 1.296±0.521; Catestatin: 2.899±1.05 [ln ms²], p=0.05).

### DISCUSSION

Mice deficient in endogenous peptide, CST have increased heart rate, decreased HRV and altered autonomic heart rate modulation in comparison to WT control mice. The widening of HRV value by CST suggests its potential utility in the cardiovascular area. The findings of improved HRV by CST in mice seem to corroborate our findings in modification of human autonomic activity by CST (14). At baseline, standard deviation of N-N intervals, total power, VLF and LF power of HRV were lower. Spectral analysis of HR signals in the frequency domain revealed that HR variability of KO mice were decreased in the VLF band indicating general modifications of neurohumoral This observation mends well with the findings in human subjects having hypertension associated with insulin-resistance syndrome, and hypertension has been a distinct feature in the current knock-out mice model (13) and in human genetic hypertension involving CgA (12). It also strengthens the arguments concerning increased cardiovascular risk by impairments in total and VLF power of heart rate in KO mice and the important role CGA peptide, catestatin plays in the stability of HRV.

The LF component corresponds mainly to sympathetic modulation and partially to parasympathetic modulation, whereas the HF component represents only parasympathetic modulation that was also assessed by short-term indices of Lorentz' plot (S1). The quotient of (S1/S2) was lower in the KO group in the basal stage, suggesting dampened sympatho-vagal balance and favoring parasympathetic modulation. Frequency power also showed diminished LF responses at baseline in KO group, which shows that direct sympathetic modulation of cardiac activity decreases. The finding of reduced LF power in KO mice runs contrary to usual expectation and to the proposed view of enhanced sympathetic drive triggering elevation of systolic and diastolic pressure. However, it is known that enhanced sympathetic drive may not necessarily reflect in altered LF or any other spectral power. This is even more evident in case of CHF, whereby enhanced sympathetic drive does not translate into increased LF power, as reported by Guzzetti et al. (35). Since KO mice are diseased or prone to compromise of their cardiovascular system like CHF patient, and thereby may fail to reveal its enhanced LF properties. The findings in KO mice are very similar to the unhealthy human subjects, in that the total power is reduced and the largest RR intervals are lost, as in CHF.

### EXAMPLE 4

### Catestatin Acts as an Angiogenic Peptide

### MATERIALS AND METHODS

CST peptide and antiserum. Please see Example 2 for the details.

Cornea Neovascularisation Assay. Pellets containing 500 ng Catestatin were implanted in C57/BL/6J mice (age of 8 weeks) as described (36). On postoperative day 7 mice received an intravenous injection of 500 μg BS1 lectin conjugated to FITC (Vector Laboratories). After euthanasia, enucleated eyes were fixed in 1% paraformaldehyde, corneas dissected and examined by fluorescence microscopy.

Mouse hind-limb ischemia model. Male C57BL/6 wild-type mice at the age of 12 months were subjected to unilateral hind-limb surgery. Briefly, the left femoral artery was exposed, ligated with 5-0 silk ligatures, and excised. Mice were injected with saline or 10 μg catestatin into thigh and calf muscles after operation and every other day for weeks 1 and 2 and 2 times per week for weeks 3 and 4. All further measurements and analyses were performed by investigators blinded to the treatment of the animals.

Blood flow measurement. Blood flow measurements were performed using a laser Doppler perfusion image (LDPI) analyzer (Moor Instruments, USA) as described. To minimize data variables attributable to ambient light and temperature animals were kept on a heating plate at 37° C. before measurement and blood perfusion is expressed as the LDPI index representing the ratio of left (=operated, ischemic leg) versus right (=not-operated, not-ischemic leg) limb blood flow. A ratio of 1 before operation indicates equal blood perfusion of both legs, whereas after femoral artery excision this ratio drops to 0.26, indicating severe attenuation of leg blood supply in the operated leg.

Alkaline phosphatase staining and Immunohistochemistry. For tissue staining, mice were sacrificed and ischemic limb tissues were retrieved after 4 weeks. Specimens were fixed in 10% (v/v) buffered formaldehyde, dehydrated with graded ethanol series and embedded in paraffin. Alternatively, fresh tissue was embedded in OCT compound (TISSUE-TEK®, Sakura Finetek) and snap-frozen in liquid nitrogen. Tissues were sliced into 5-μm sections. Vascular endothelial cells were identified by alkaline phosphatase staining and for assessment of artery/arteriole density sections were stained with a mouse monoclonal alpha-smooth muscle actin antibody (Pharmingen) as described (36). Adductor muscle samples of each leg were divided into 2 parts and capillaries (alkaline phosphatase positive) and arteries (alpha-smooth muscle actin positive) were counted in five sections of each part and are expressed as capillary and arteriole density per mm².

Statistical Analysis. Statistical analyses were done as described in Example 1.

### RESULTS

### In-vivo Studies

### 1. Cornea Neovascularization Assay

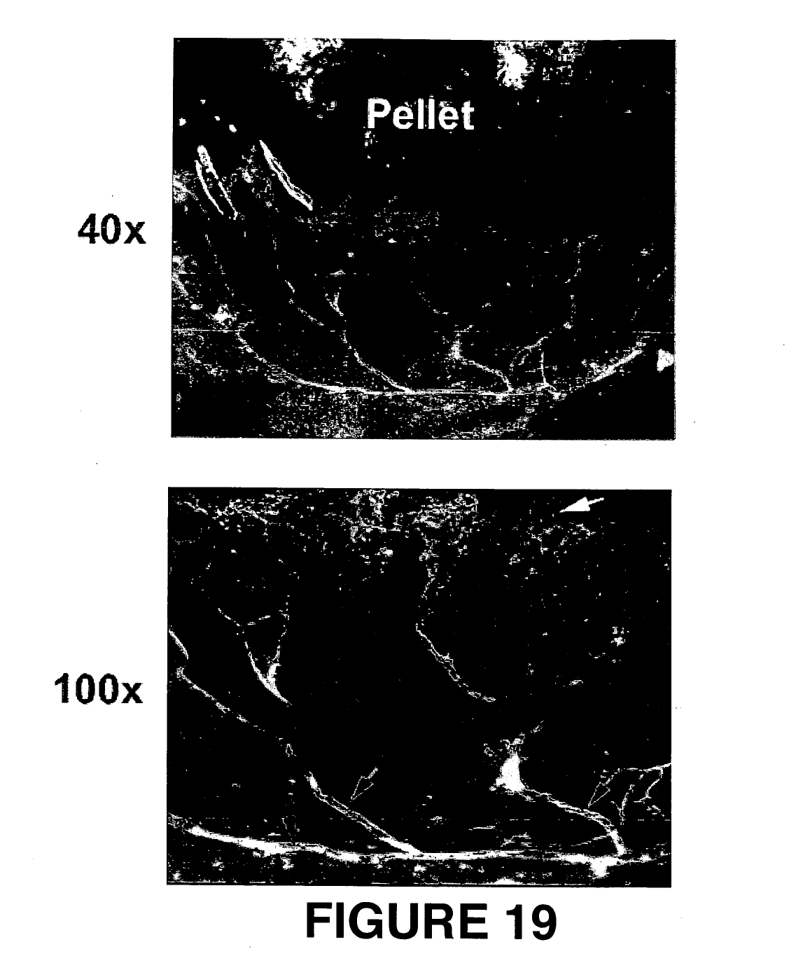

To evaluate if catestatin induces angiogenesis also in-vivo a cornea neovascularization assay was performed. Catestatin induced growth of arteries out of the limbus artery leading to a capillary network around the pellet. Also vessels reaching the limbus vein and presumably representing veins were observed (FIG. 19).

### 2. Hind-Limb Ischemia Model

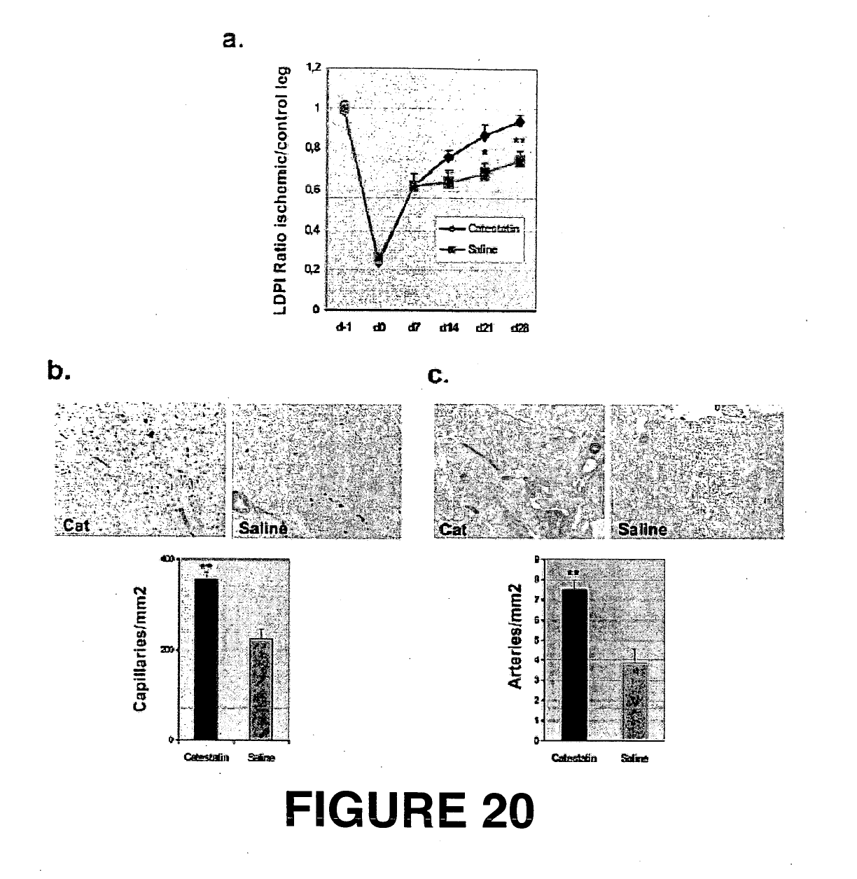

Intra-muscular injections of catestatin peptide into the ischemic limb improved blood perfusion as measured by LDPI. Perfusion ratio of control vs. ischemic limb improved in both groups, however 4 wks after induction of ischemia LDPI ratio was 0.76±0.04 in saline injected mice and 0.94±0.03 in catestatin treated animals (n=10 mice each group, P<0.01 saline vs. cat; FIG. 20). Also after 3 weeks catestatin treated animals showed significantly better perfusion ratios than saline treated mice. To determine blood vessel density in ischemic muscle immunohistochemistry was performed for alkaline phosphatase staining (detecting ECs) and for alpha smooth muscle actin (detecting pericytes/smooth muscle cells). We observed significantly higher densities of capillaries (alkaline phosphatase positive vessels) in catestatin treated mice 4 weeks after surgery (capillaries/mm²: catestatin 356±17 and saline 227±19; n=10 each group, P<0.01). Additionally we also observed increased density of alpha smooth muscle actin positive vessels after catestatin treatment (alpha-smooth muscle actin positive vessels/mm²: catestatin 7.5±0.5 and saline 3.9±0.7; n=10 each group, P<0.01). This finding of increased density of arterioles/arteries after CST treatment is consistent of induction also of arteriogenesis by this peptide.

### DISCUSSION

Since the initial observation that the peptide catestatin, which is derived from CgA, inhibits catecholamine secretion from chromaffin cells (6) several further biological effects of this molecule have been reported. Catestatin was shown to induce histamine release from mast cells (37) and increases this factor substantially in the circulation (38). Furthermore, catestatin exerts anti-microbial activity in-vitro (39) and in cutaneous wounds where it was up-regulated in fibroblasts (40). In Langendorff-perfused rat hearts and the avascular frog heart catestatin decreased cardiac contractility induced by isoproterenol and endothelin-1 (41, 42). This mechanism was dependent on beta2-adrenergic receptors as well as Gi/o protein-nitric oxide-cGMP signaling mechanisms and these findings might indicate that catestatin is able to protect the heart against excessive sympatho-chromaffin over-activation.

We recently observed that catestatin induces directed migration of human blood monocytes and therefore might act as an inflammatory cytokine (43). It also has been shown before that the precursor of catestatin, CgA is up-regulated by hypoxia in the brain (44). As inflammation and hypoxia usually are accompanied or followed by increased blood vessel generation we tested if catestatin exerts effects on ECs in-vitro and angiogenesis in-vivo. We found that catestatin exerts several direct effects on ECs including EC migration and proliferation. Blockade by a neutralizing antibody indicates specificity of observed effects. Mechanistically, catestatin induced activation of MAPK signaling pathway in ECs and inhibition of ERK inhibited observed effects indicating that activation of MAPK in ECs mediates catestatin induced effects like reported before for other angiogenic cytokines like VEGF or bFGF. Inhibition of EC chemotaxis by pertussis toxin indicates involvement of G-proteins in this observed effect.

Beside its effects on ECs catestatin also showed effects on EPCs like EPC chemotaxis and incorporation of these cells into vascular structures in-vitro. These findings indicate that this peptide also might induce post-natal vasculogenesis. It has to be shown if catestatin also induces vasculogenesis in-vivo in animal models.

In-vivo catestatin induced angiogenesis in the mouse cornea neovascularization assay and angiogenesis and arteriogenesis in the hind-limb ischemia model. Serial measurements of blood perfusion indicates that catestatin indeed was able to increase perfusion to levels before ligation of the femoral artery yielding a significant better value compared to saline injected animals. These observations indicate that catestatin indeed is a novel angiogenic cytokine that exerts direct effects on ECs.

Notably, it has been shown that another peptide derived from CgA, vasostatin, inhibited angiogensis induced by vascular endothelial growth factor (VEGF). Vasostatin blocked VEGF-induced MAPK activation in ECs and inhibited angiogenesis exerted by this factor in-vivo in the matrigel assay. The main difference between our findings on angiogenesis induced by catestatin and the inhibitory effect of vasostatin on VEGF-induced blood vessel formation is the concentration of the respective peptide. Whereas vasostatin-induced effects were found at a concentration of 330 nM, we observed a maximal function of catestatin at 1 nM, i.e. two magnitudes of concentration lower. At higher concentrations of catestatin (100 nM) angiogenic effects were still observed in-vitro (EC migration, proliferation and in-vitro matrigel assay) indicating that CgA-derived peptides do not block angiogenesis un-selectively at higher concentrations. The biological consequences of these, in the initial view contradictory findings-inhibitory versus stimulatory effects on angiogenesis by two different peptides derived from the same molecule CgA- have to be determined. Nevertheless it is conceivable that dependent on local processing of CgA, on local concentrations of peptides and the responsiveness of the target cell, CgA-derived peptides might act as angiogenic or anti-angiogenic molecules.

It is also notable that secretoneurin, a peptide derived from another member of the chromogranin/secretogranin family of acidic storage proteins of vesicles in neuro-endocrine cells, secretogranin II, induces angiogenesis (36). Therefore, catestatin and secretoneurin might be promising novel candidates in the therapy of diseases like limb or myocardial ischemia.

### EXAMPLE 5

### CST is a New Player in the Physiological Regulation of Glucose Production and Insulin Clearance

### BACKGROUND

CST raises blood glucose level in both CgA-knockout (CgA-KO) and wild type (WT) mice but suppress glucose level in high fat fed insulin resistant hyperglycemic mice. It possible that CST reduces blood glucose level in all diabetic models and diabetic patients. These disparate results can be reconciled by assigning a critical role of eNOS and nitric oxide (NO) on hepatic glucose metabolism. A number of publications in literature demonstrated that NO inhibits glucose production and glycogen synthesis but stimulates glycogenolysis. CST, on the other hand, reduces p-AMPK and p-eNOS signals (see our results), elevates gluconeogenesis but, may reduce glycogenolysis (effects of low NO level and AMPK activity levels).

Chronic intake of nicotine through smoking keeps catecholamine pathway active and blood pressure and pre- as well as postprandial glucose levels higher than non-smokers. Thus, repeated assault on the homeostatic mechanism with higher blood glucose level and blood pressure end up producing increased risk of metabolic disorders in the smokers. If the smokers are diabetic, their risk in cardiovascular dysfunction is further increased. In this set up, treatment with CST, which antagonizes nicotine induced cardiovascular dysfunction, will provide double benefit by reducing glucose level in diabetic patients. We believe that CST will do that by inhibition of glycogenolysis and enhancement of insulin-stimulated glycogen production in both liver and muscle.

### METHODS AND MATERIALS

Adult WT (C57BL/6) and CgA-KO mice with mixed genetic background (129svJ×C57BL/6) were used in this study. Both WT and KO mice were generated from the original founder carrying mixed genotype (50% 129svJ: 50% C57BL/6) and were maintained by brother/sister mating. Hypertensive CGA-KO mice will serve as a model for hyper insulin sensitivity. WT mice were fed 60% high fat diet (HFD) for 20 weeks. Body weight and blood glucose levels were monitored and glucose and insulin tolerance testa were performed to determine status of insulin resistance. After 20 weeks of HFD feeding mice were injected IP with catestatin (40 μg/gm body weight) twice daily for 7 days. On the 8^{th} day, 12 hours fasted mice were subjected to glucose tolerance tests to determine their insulin sensitivity. Tissues of WT and CgA-KO mice are analyzed AMPK and eNOS signalling by immunoblotting.

### RESULTS AND DISCUSSION

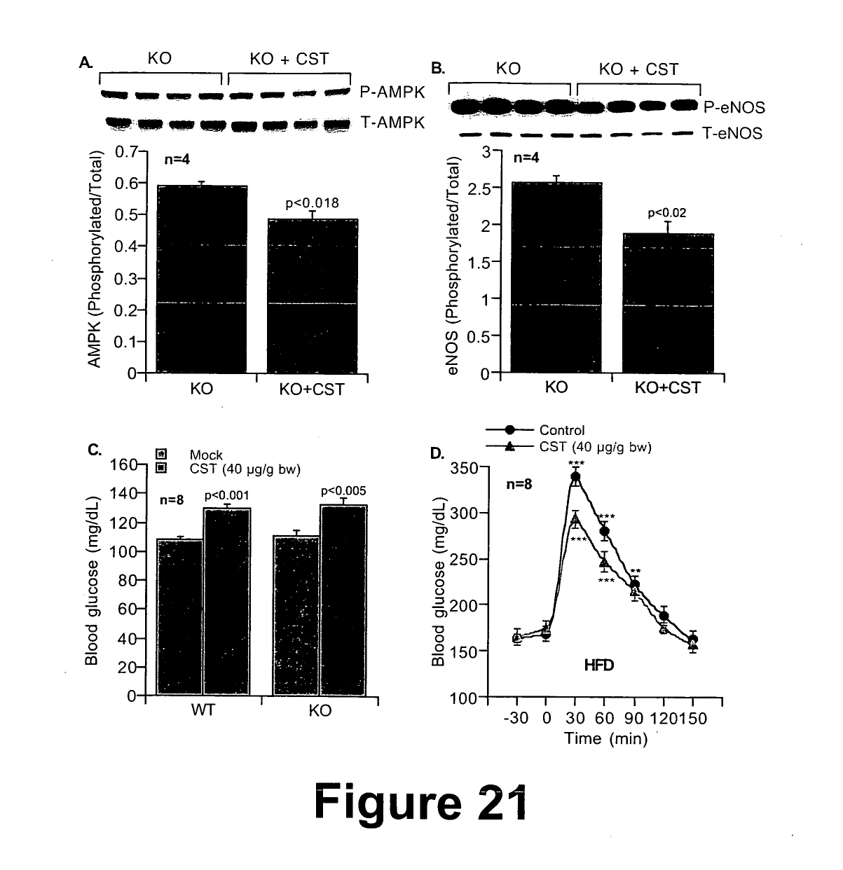

As shown in FIG. 21, CST treatment reduced pAMPK (A), eNOS (B) signals in CgA-KO mice, raised blood glucose levels in both WT and CgA-KO mice (C) but reduced peak blood glucose level in HFD fed insulin resistant mice (D).

Treatment of insulin-resistant HFD mice with CST alone reduced blood glucose level by 15-20% as seen by glucose tolerance tests (FIG. 21D). This result was surprising because CST seemed to have dual and opposite effects; hyperglycemic in WT mice whereas hypoglycemic in the HFD-induced insulin resistant model. We believe that CST inhibits hepatic glycogenolysis by reducing cAMP and NO levels and this inhibition lowers glucose level significantly in diabetic models that are already hyperglycemic where gluconeogenic effect of CST does not make significant additional contribution

### CONCLUSION

We have discovered that CST is a new player in the physiological regulation of glucose production and insulin clearance, the two key factors whose dysfunctions lead to metabolic syndrome. Our results suggest that CST could be a practical therapy against insulin resistance and diabetes, particularly for diabetic patients who are also smokers and have hypertension. Antinicotinic CST will reduce blood pressure and glucose levels at the same time in those patients.

### REFERENCES

1. Winkler, H., and Fischer-Colbrie, R. 1992. The chromogranins A and B: the first 25 years and future perspectives. Neuroscience 49:497-528.
2. Taupenot, L., Harper, K. L., and O'Connor, D. T. 2003. Mechanisms of disease: The chromogranin-secretogranin family. New Engl J Med 348:1134-1149.
3. Montero-Hadjadje, M., Vaingankar, S., Elias, S., Tostivint, H., Mahata, S. K., and Anouar, Y. 2008. Chromogranins A and B and secretogranin II: evolutionary and functional aspects. Acta Physiol (Oxf) 192:309-324.
4. Tatemoto, K., Efendic, S., Mutt, V., Makk, G., Feistner, G. J., and Barchas, J. D. 1986. Pancreastatin, a novel pancreatic peptide that inhibits insulin secretion. Nature 324:476-478.
5. Aardal, S., Helle, K. B., Elsayed, S., Reed, R. K., and Serck-Hanssen, G. 1993. Vasostatins, comprising the N-terminal domain of chromogranin A, suppress tension in isolated human blood vessel segments. J Neuroendocrinol 5:405-412.
6. Mahata, S. K., O'Connor, D. T., Mahata, M., Yoo, S. H., Taupenot, L., Wu, H., Gill, B. M., and Parmer, R. J. 1997. Novel autocrine feedback control of catecholamine release. A discrete chromogranin A fragment is a noncompetitive nicotinic cholinergic antagonist. J Clin Invest 100:1623-1633.
7. Mahata, S. K., Mahata, M., Parmer, R. J., and O'Connor, D. T. 1999. Desensitization of catecholamine release: The novel catecholamine release-inhibitory peptide catestatin (chromogranin A₃₄₄₋₃₆₄) acts at the receptor to prevent nicotinic cholinergic tolerance. J Biol Chem 274:2920-2928.
8. Mahata, S. K., Mahata, M., Wakade, A. R., and O'Connor, D. T. 2000. Primary structure and function of the catecholamine release inhibitory peptide catestatin (chromogranin A344-364): Identification of amino acid residues crucial for activity. Mol Endocrinol 14:1525-1535.
9. Wen, G., Mahata, S. K., Cadman, P., Mahata, M., Ghosh, S., Mahapatra, N. R., Rao, F., Stridsberg, M., Smith, D. W., Mahboubi, P., et al. 2004. Both rare and common polymorphisms contribute functional variation at CHGA, a regulator of catecholamine physiology. Am J Hum Genet 74:197-207.
10. Mahata, S. K. 2004. Catestatin—The catecholamine release inhibitory peptide: A structural and functional overview. Curr Med Chem—Immun Endoc & Metab Agents 4:221-234.
11. Mahata, S. K., Mahata, M., Wen, G., Wong, W. B., Mahapatra, N. R., Hamilton, B. A., and O'Connor, D. T. 2004. The catecholamine release-inhibitory “catestatin” fragment of chromogranin a: naturally occurring human variants with different potencies for multiple chromaffin cell nicotinic cholinergic responses. Mol Pharmacol 66:1180-1191.
12. O'Connor, D. T., Kailasam, M. T., Kennedy, B. P., Ziegler, M. G., Yanaihara, N., and Parmer, R. J. 2002. Early decline in the catecholamine release-inhibitory peptide catestatin in humans at genetic risk of hypertension. J Hypertens 20:1335-1345.
13. Mahapatra, N. R., O'Connor, D. T., Vaingankar, S. M., Sinha Hikim, A. P., Mahata, M., Ray, S., Staite, E., Wu, H., Gu, Y., Dalton, N., et al: 2005. Hypertension from targeted ablation of chromogranin A can be rescued by the human ortholog. J Clin Invest 115:1942-1952.
14. Rao, F., Wen, G., Gayen, J. R., Das, M., Vaingankar, S. M., Rana, B. K., Mahata, M., Kennedy, B. P., Salem, R. M., Stridsberg, M., et al. 2007. Catecholamine release-inhibitory peptide catestatin (chromogranin A (352-372)): naturally occurring amino acid variant Gly364Ser causes profound changes in human autonomic activity and alters risk for hypertension. Circulation 115:2271-2281.
15. Helle, K. B., Corti, A., Metz-Boutigue, M. H., and Tota, B. 2007. The endocrine role for chromogranin A: A prohormone for peptides with regulatory properties. Cell Mol Life Sci.
16. Mahata, S. K., Mahapatra, N. R., Mahata, M., Wang, T. C., Kennedy, B. P., Ziegler, M. G., and O'Connor, D. T. 2003. Catecholamine secretory vesicle stimulus-transcription coupling in vivo. Demonstration by a novel transgenic promoter/photoprotein reporter and inhibition of secretion and transcription by the chromogranin A fragment catestatin. J Biol Chem 278:32058-32067.
17. Takiyyuddin, M. A., Cervenka, J. H., Sullivan, P. A., Pandian, M. R., Parmer, R. J., Barbosa, J. A., and O'Connor, D. T. 1990. Is physiologic sympathoadrenal catecholamine release exocytotic in humans? Circulation 81:185-195.
18. Koeslag, J. H., Saunders, P. T., and Wessels, J. A. 1999. The chromogranins and the counter-regulatory hormones: do they make homeostatic sense? J Physiol 517 (Pt 3):643-649.
19. Ceconi, C., Ferrari, R., Bachetti, T., Opasich, C., Volterrani, M., Colombo, B., Parrinello, G., and Corti, A. 2002. Chromogranin A in heart failure; a novel neurohumoral factor and a predictor for mortality. Eur Heart J 23:967-974.
20. Pieroni, M., Corti, A., Tota, B., Cumis, F., Angelone, T., Colombo, B., Cerra, M. C., Bellocci, F., Crea, F., and Maseri, A. 2007. Myocardial production of chromogranin A in human heart: a new regulatory peptide of cardiac function. Eur Heart J 28:1117-1127.
21. Takiyyuddin, M. A., Baron, A. D., Cervenka, J. H., Barbosa, J. A., Neumann, H. P., Parmer, R. J., Sullivan, P. A., and O'Connor, D. T. 1991. Suppression of chromogranin-A release from neuroendocrine sources in man: pharmacological studies. J Clin Endocrinol Metab 72:616-622.
22. Takiyyuddin, M. A., Parmer, R. J., Kailasam, M. T., Cervenka, J. H., Kennedy, B., Ziegler, M. G., Lin, M. C., Li, J., Grim, C. E., Wright, F. A., et al. 1995. Chromogranin A in human hypertension. Influence of heredity. Hypertension 26:213-220.
23. Angelone, T., Goumon, Y., Cerra, M. C., Metz-Boutigue, M. H., Aunis, D., and Tota, B. 2006. The emerging cardioinhibitory role of the hippocampal cholinergic neurostimulating peptide. J Pharmacol Exp Ther 318:336-344.
24. Georget, M., Mateo, P., Vandecasteele, G., Lipskaia, L., Defer, N., Hanoune, J., Hoerter, J., Lugnier, C., and Fischmeister, R. 2003. Cyclic AMP compartmentation due to increased cAMP-phosphodiesterase activity in transgenic mice with a cardiac-directed expression of the human adenylyl cyclase type 8 (AC8). Faseb J 17:1380-1391.
25. Cappello, S., Angelone, T., Tota, B., Pagliaro, P., Penna, C., Rastaldo, R., Corti, A., Losano, G. A., and Cerra, M. C. 2007. Human recombinant Chromogranin A-derived Vasostatin-1 Mimics Preconditioning via an Adenosine/Nitric Oxide Signalling Mechanism. Am J Physiol Heart Circ Physiol 293:H719-727.
26. Vittone, L., Mundina-Weilenmann, C., Mattiazzi, A., and Cingolani, H. 1994. Physiologic and pharmacologic factors that affect myocardial relaxation. J Pharmacol Toxicol Methods 32:7-18.
27. Brar, B. K., Jonassen, A. K., Egorina, E. M., Chen, A., Negro, A., Perrin, M. H., Mjos, O. D., Latchman, D. S., Lee, K. F., and Vale, W. 2004. Urocortin-II and urocortin-III are cardioprotective against ischemia reperfusion injury: an essential endogenous cardioprotective role for corticotropin releasing factor receptor type 2 in the murine heart. Endocrinology 145:24-35.
28. Cerra, M. C., and Pellegrino, D. 2007. Cardiovascular cGMP-generating systems in physiological and pathological conditions. Curr Med Chem 14:585-599.
29. Abbott, D., and Lodola, A. 1992. The effects of isoproterenol on the isolated perfused rat heart. Res Commun Chem Pathol Pharmacol 76:125-128.
30. Asimakis, G. K., Inners-McBride, K., Conti, V. R., and Yang, C. J. 1994. Transient beta adrenergic stimulation can precondition the rat heart against postischaemic contractile dysfunction. Cardiovasc Res 28:1726-1734.
31. Brunner, F., Bras-Silva, C., Cerdeira, A. S., and Leite-Moreira, A. F. 2006. Cardiovascular endothelins: essential regulators of cardiovascular homeostasis. Pharmacol Ther 111:508-531.
32. Bigger, J. T., Jr., Fleiss, J. L., Steinman, R. C., RoInitzky, L. M., Schneider, W. J., and Stein, P. K. 1995. RR variability in healthy, middle-aged persons compared with patients with chronic coronary heart disease or recent acute myocardial infarction. Circulation 91:1936-1943.
33. Deftos, L. J., Woloszczuk, W., Krisch, I., Horvat, G., Ulrich, W., Neuhold, N., Braun, O., Reiner, A., Srikanta, S., and Krisch, K. 1988. Medullary thyroid carcinomas express chromogranin A and a novel neuroendocrine protein recognized by monoclonal antibody HISL-19. Am J Med 85:780-784.
34. Gehrmann, J., Hammer, P. E., Maguire, C. T., Wakimoto, H., Triedman, J. K., and Berul, C. I. 2000. Phenotypic screening for heart rate variability in the mouse. Am J Physiol Heart Circ Physiol 279:H733-740.
35. Guzzetti, S., Magatelli, R., Borroni, E., and Mezzetti, S. 2001. Heart rate variability in chronic heart failure. Auton Neurosci 90:102-105.
36. Kirchmair, R., Gander, R., Egger, M., Hanley, A., Silver, M., Ritsch, A., Murayama, T., Kaneider, N., Sturm, W., Kearny, M., et al. 2004. The neuropeptide secretoneurin acts as a direct angiogenic cytokine in vitro and in vivo. Circulation 109:777-783.
37. Kruger, P. G., Mahata, S. K., and Helle, K. B. 2003. Catestatin (CgA344-364) stimulates rat mast cell release of histamine in a manner comparable to mastoparan and other cationic charged neuropeptides. Regul Pept 114:29-35.
38. Dao, T. T., Kailasam, M. T., Parmer, R. J., Le, H. V., Le Verge, R., Kennedy, B. P., Ziegler, G., Insel, P. A., Wright, F. A., and O'Connor, D. T. 1998. Expression of altered alpha2-adrenergic phenotypic traits in normotensive humans at genetic risk of hereditary (essential) hypertension. J Hypertens 16:779-792.
39. Briolat, J., Wu, S. D., Mahata, S. K., Gonthier, B., Bagnard, D., Chasserot-Golaz, S., Helle, K. B., Aunis, D., and Metz-Boutigue, M. H. 2005. New antimicrobial activity for the catecholamine release-inhibitory peptide from chromogranin A. Cell Mol Life Sci 62:377-385.
40. Radek, K. A., Lopez-Garcia, B., Hupe, M., Niesman, I. R., Elias, P. M., Taupenot, L., Mahata, S. K., O'Connor, D. T., and Gallo, R. L. 2008. The neuroendocrine peptide catestatin is a cutaneous antimicrobial and induced in the skin after injury. J Invest Dermatol 128:1525-1534.
41. Mazza, R., Gattuso, A., Mannarino, C., Brar, B. K., Barbieri, S. F., Tota, B., and Mahata, S. K. 2008. Catestatin (chromogranin A344-364) is a novel cardiosuppressive agent: inhibition of isoproterenol and endothelin signaling in the frog heart. Am. J Physiol Heart Circ Physiol 295:H113-122.
42. Angelone, T., Quintieri, A. M., Brar, B. K., Limchaiyawat, P. T., Tota, B., Mahata, S. K., and Cerra, M. C. 2008. The Antihypertensive Chromogranin A-derived Peptide Catestatin As a Novel Endocrine/Paracrine Modulator of Cardiac Function: Inotropic and Lusitropic Actions on the Rat Heart. Endocrinology 149:4780-4793.
43. Egger, M., Beer, A. G., Theurl, M., Schgoer, W., Hotter, B., Tatarczyk, T., Vasiljevic, D., Frauscher, S., Marksteiner, J., Patsch, J. R., et al. 2008. Monocyte migration: a novel effect and signaling pathways of catestatin. Eur J Pharmacol 598:104-111.
44. Wade-Martins, R., White, R. E., Kimura, H., Cook, P. R., and James, M. R. 2000. Stable correction of a genetic deficiency in human cells by an episome carrying a 115 kb genomic transgene. Nat Biotechnol 18:1311-1314.

What is claimed is:

1. An isolated peptide called catestatin comprising the amino acid sequence CHGA₃₅₂₋₃₇₂ or a sequence 80-99.9% homologous to CHGA₃₅₂₋₃₇₂.

2. An isolated polypeptide comprising the amino acid variation of Gly364 to Ser within the catestatin domain, which is designated as Gly364Ser.

3. An isolated polypeptide comprising the amino acid variation of Pro370 to Leu within the catestatin domain, which is designated as Pro370Leu.

4. A pharmaceutical composition comprising a pharmaceutically effective amount of the polypeptide of claim 1.

5. A pharmaceutical composition comprising a pharmaceutically effective amount of the polypeptide of claim 2.

6. A pharmaceutical composition comprising a pharmaceutically effective amount of the polypeptide of claim 3.

7. An isolated nucleic acid which encodes for the amino acid sequence for CHGA₃₅₂₋₃₇₂, or Gly364Ser, or Pro370Leu.

8. A polyclonal or monoclonal antibody against catestatin

9. A variant or antagonist of catestatin

10. Any chemical modification of catestatin such as, acylation, phosphorylation, glycosylation, acetylation, amidation and nitrosylation.

11. A method of treatment comprising administration of pharmaceutical composition of catestatin or its antagonists or its chemically modified form or its variants such as, Gly364Ser and Pro370Leu or a peptide 80-99.9% homologous to catestatin to human subjects or mammals in general
a) who is at risk of or shows symptoms of hypertension, cardiovascular disease or metabolic disease such as, diabetes and glucose intolerance;
b) to change blood glucose or insulin levels or inflammatory status;
c) to minimize nicotine or tobacco addiction

12. A method of diagnosing an individual who is suffering from hypertension or who is at risk of developing hypertension comprising:
a) providing an individual who is suspected to be suffering from hypertension, cardiovascular disease or metabolic disease; or who is at risk of developing hypertension, cardiovascular disease or metabolic disease;
b) taking a blood sample from said individual;
c) determining the level of catestatin (CHGA₃₅₂₋₃₇₂) or its variants Gly364Ser or
Pro370Leu in said blood sample;
d) correlating said level to levels found in population of healthy subjects; and
e) identifying whether or not said individual is suffering from hypertension, cardiovascular disease or metabolic disease; or is at risk of developing hypertension, cardiovascular disease or metabolic disease.
